SEQUENCE LISTING (87 sequences)

SEQ ID NO: 1 - protein, 22 aa, Homo sapiens
AVDLGNIWRFPYICYQNGGGAF

SEQ ID NO: 2 - protein, 22 aa, Homo sapiens
LISSFTDQLPWTSCKNSWNTGN

SEQ ID NO: 3 - protein, 22 aa, Homo sapiens
PWTSCKNSWNTGNCTNYFAQDN

SEQ ID NO: 4 - protein, 22 aa, Homo sapiens
QAFAQYTDKHGEVCPAGWKPGS

SEQ ID NO: 5 - protein, 21 aa, Homo sapiens
YMPKKATELKHLQCLEEELKP

SEQ ID NO: 6 - protein, 22 aa, Homo sapiens
IVLELKGSETTFMCEYADETAT

SEQ ID NO: 7 - protein, 22 aa, Homo sapiens
ATIVEFLNRWITFCQSIISTLT

SEQ ID NO: 8 - protein, 5 aa, Homo sapiens
RYCLL

SEQ ID NO: 9 - protein, 19 aa, Homo sapiens
DISQCGRRDCAVKPCQSDE

SEQ ID NO: 10 - protein, 5 aa, Homo sapiens
YICYQ

SEQ ID NO: 11 - protein, 5 aa, Homo sapiens
TSCKN

SEQ ID NO: 12 - protein, 9 aa, Homo sapiens
HGEVCPAGW

SEQ ID NO: 13 - protein, 9 aa, Homo sapiens
HGEVCPAGW

SEQ ID NO: 14 - protein, 5 aa, Homo sapiens
EVCPA

SEQ ID NO: 15 - protein, 5 aa, Homo sapiens
LQCLE

SEQ ID NO: 16 - protein, 5 aa, Homo sapiens
FMCEY

SEQ ID NO: 17 - protein, 5 aa, Homo sapiens
TFCQS

SEQ ID NO: 18 - protein, 15 aa, Homo sapiens
SQCGRRDCAVKPCQS

SEQ ID NO: 19 - protein, 18 aa, Homo sapiens
PTLYNVSLVMSDTAGTCY

SEQ ID NO: 20 - protein, 18 aa, Mus musculus
PTLYNVSLIMSDTGGTCY

SEQ ID NO: 21 - protein, 18 aa, Homo sapiens
PTHVNVSVVMAEVDGTCY

SEQ ID NO: 22 - protein, 19 aa, Mus musculus
PTNVSVVSVIMSEGDGICY

SEQ ID NO: 23 - protein, 7 aa, Homo sapiens
DTAGTCY

SEQ ID NO: 24 - protein, 7 aa, Homo sapiens
DTGGTCY

SEQ ID NO: 25 - protein, 4 aa, Homo sapiens
GTCY

SEQ ID NO: 26 - protein, 11 aa, Artificial Sequence
  Synthetic peptide sequence
YGRKKRRQRRR

SEQ ID NO: 27 - protein, 12 aa, Artificial Sequence
  Synthetic peptide sequence
YGRKKRRQQRRR

SEQ ID NO: 28 - protein, 7 aa, Artificial Sequence
  Synthetic peptide sequence
YGRKKRR

SEQ ID NO: 29 - protein, 13 aa, Artificial Sequence
  Synthetic peptide sequence
GRKKRRQRRRPPQ

SEQ ID NO: 30 - protein, 16 aa, Artificial Sequence
  Synthetic peptide sequence
RQIKIWFQNRRMKWKK

SEQ ID NO: 31 - protein, 19 aa, Artificial Sequence
  Synthetic peptide sequence
VKRGLKLRHVRPRVTRMDV

SEQ ID NO: 32 - protein, 24 aa, Artificial Sequence
  Synthetic peptide sequence
RRIRPRPPRLPRPRPRPLPFPRPG

SEQ ID NO: 33 - protein, 18 aa, Artificial Sequence
  Synthetic peptide sequence
RGGRLSYSRRRFSTSTGR

SEQ ID NO: 34 - protein, 28 aa, Artificial Sequence
  Synthetic peptide sequence
RGGRLSYSRRRFSTSTGRWSQPKKKRKV

SEQ ID NO: 35 - protein, 34 aa, Artificial Sequence
  Synthetic peptide sequence
DAATATRGRSAASRPTQRPRAPARSASRPRRPVQ

SEQ ID NO: 36 - protein, 17 aa, Artificial Sequence
  Synthetic peptide sequence
NAKTRRHERRRKLAIER

SEQ ID NO: 37 - protein, 27 aa, Artificial Sequence
  Synthetic peptide sequence
GWTLNSAGYLLGKINLKALAALAKKIL

SEQ ID NO: 38 - protein, 18 aa, Artificial Sequence
  Synthetic peptide sequence
KLALKLALKALKAALKLA

SEQ ID NO: 39 - protein, 18 aa, Artificial Sequence
  Synthetic peptide sequence
LLIILRRRIRKQAHAHSK

SEQ ID NO: 40 - protein, 16 aa, Artificial Sequence
  Synthetic peptide sequence
AAVALLPAVLLALLAP

SEQ ID NO: 41 - protein, 24 aa, Artificial Sequence
  Synthetic peptide sequence
LGTYTQDFNKFHTFPQTAIGVGAP

SEQ ID NO: 42 - protein, 27 aa, Artificial Sequence
  Synthetic peptide sequence
GALFLGFLGAAGSTMGAWSQPKKKRKV

SEQ ID NO: 43 - protein, 17 aa, Artificial Sequence
  Synthetic peptide sequence
GALFLGFLGAAGSTMGA

SEQ ID NO: 44 - protein, 21 aa, Artificial Sequence
  Synthetic peptide sequence
TRSSRAGLQFPVGRVHRLLRK

SEQ ID NO: 45 - protein, 21 aa, Artificial Sequence
  Synthetic peptide sequence
KETWWETWWTEWSQPKKKRKV

SEQ ID NO: 46 - protein, 23 aa, Artificial Sequence
  Synthetic peptide sequence
GIGKFLHSAKKFGKAFVGEIMNS

SEQ ID NO: 47 - protein, 22 aa, Artificial Sequence
  Synthetic peptide sequence
MVTVLFRRLRIRRACGPPRVRV

SEQ ID NO: 48 - protein, 17 aa, Artificial Sequence
  Synthetic peptide sequence
CSIPPEVKFNKPFVYLI

SEQ ID NO: 49 - protein, 6 aa, Artificial Sequence
  Synthetic peptide sequence
PFVYLI

SEQ ID NO: 50 - protein, 18 aa, Artificial Sequence
  Synthetic peptide sequence
LLIILRRRIRKQAHAHSK

SEQ ID NO: 51 - protein, 28 aa, Artificial Sequence
  Synthetic peptide sequence
MVKSKIGSWILVLFVAMWSDVGLCKKRP

SEQ ID NO: 52 - protein, 22 aa, Artificial Sequence
  Synthetic peptide sequence
MVRRFLVTLRIRRACGPPRVRV

SEQ ID NO: 53 - protein, 28 aa, Artificial Sequence
  Synthetic peptide sequence
LSTAADMQGVVTDGMASGLDKDYLKPDD

SEQ ID NO: 54 - protein, 26 aa, Artificial Sequence
  Synthetic peptide sequence
DPKGDPKGVTVTVTVTVTGKGDPKPD

SEQ ID NO: 55 - protein, 16 aa, Artificial Sequence
  Synthetic peptide sequence
YTAIAWVKAFIRKLRK

SEQ ID NO: 56 - protein, 17 aa, Artificial Sequence
  Synthetic peptide sequence
IAWVKAFIRKLRKGPLG

SEQ ID NO: 57 - protein, 20 aa, Artificial Sequence
  Synthetic peptide sequence
GLWRALWRLLRSLWRLLWRA

SEQ ID NO: 58 - protein, 15 aa, Artificial Sequence
  Synthetic peptide sequence
SDLWEMMMVSLACQY

SEQ ID NO: 59 - protein, 9 aa, Artificial Sequence
  Synthetic peptide sequence
KGTCAGWMA

SEQ ID NO: 60 - protein, 21 aa, Artificial Sequence
  Synthetic peptide sequence
YGRKKRRQQRRRKGTCAGWMA

SEQ ID NO: 61 - protein, 17 aa, Artificial Sequence
  Synthetic peptide sequence
YGRKKKRRQQRRRGTCY

SEQ ID NO: 62 - protein, 20 aa, Artificial Sequence
  Synthetic peptide sequence
YGRKKKRRQQRRRDTGGTCY

SEQ ID NO: 63 - protein, 17 aa, Artificial Sequence
  Synthetic peptide sequence
YGRKKRRQQRRRYICYQ

SEQ ID NO: 64 - protein, 17 aa, Artificial Sequence
  Synthetic peptide sequence
YGRKKRRQQRRRTSCKN

SEQ ID NO: 65 - protein, 17 aa, Artificial Sequence
  Synthetic peptide sequence
YGRKKRRQQRRRGNCTN

SEQ ID NO: 66 - protein, 21 aa, Artificial Sequence
  Synthetic peptide sequence
YGRKKRRQQRRRHGEVCPAGW

SEQ ID NO: 67 - protein, 17 aa, Artificial Sequence
  Synthetic peptide sequence
YGRKKRRQQRRREVCPA

SEQ ID NO: 68 - protein, 17 aa, Artificial Sequence
  Synthetic peptide sequence
YGRKKRRQQRRRLQCLE

SEQ ID NO: 69 - protein, 17 aa, Artificial Sequence
  Synthetic peptide sequence
YGRKKRRQQRRRFMCEY

SEQ ID NO: 70 - protein, 17 aa, Artificial Sequence
  Synthetic peptide sequence
YGRKKRRQQRRRTFCQS

SEQ ID NO: 71 - protein, 17 aa, Artificial Sequence
  Synthetic peptide sequence
YGRKKRRQQRRRRYCLL

SEQ ID NO: 72 - protein, 27 aa, Artificial Sequence
  Synthetic peptide sequence
YGRKKRRQQRRRSQCGRRDCAVKPCQS

SEQ ID NO: 73 - protein, 8 aa, Homo sapiens
WTSCKNSW

SEQ ID NO: 74 - protein, 19 aa, Artificial Sequence
  Synthetic peptide sequence
YGRKKRRQRRRWTSCKNSW

SEQ ID NO: 75 - protein, 8 aa, Homo sapiens
TGNCTNYF

SEQ ID NO: 76 - protein, 19 aa, Artificial Sequence
  Synthetic peptide sequence
YGRKKRRQRRRTGNCTNYF

SEQ ID NO: 77 - protein, 16 aa, Homo sapiens
WTSCKNSWNTGNCTNY

SEQ ID NO: 78 - protein, 27 aa, Artificial Sequence
  Synthetic peptide sequence
YGRKKRRQRRRWTSCKNSWNTGNCTNY

SEQ ID NO: 79 - protein, 9 aa, Artificial Sequence
  Synthetic peptide sequence
KGTCYGWMA

SEQ ID NO: 80 - protein, 20 aa, Artificial Sequence
  Synthetic peptide sequence
YGRKKRRQRRRKGTCYGWMA

SEQ ID NO: 81 - protein, 8 aa, Artificial Sequence
  Synthetic peptide sequence
GTCYGWMA

SEQ ID NO: 82 - protein, 19 aa, Artificial Sequence
  Synthetic peptide sequence
YGRKKRRQRRRGTCYGWMA

SEQ ID NO: 83 - protein, 4 aa, Artificial Sequence
  Synthetic peptide sequence
GTCA

SEQ ID NO: 84 - protein, 15 aa, Artificial Sequence
  Synthetic peptide sequence
YGRKKRRQRRRGTCA

SEQ ID NO: 85 - protein, 5 aa, Artificial Sequence
  Synthetic peptide sequence
KGTCY

SEQ ID NO: 86 - protein, 16 aa, Artificial Sequence
  Synthetic peptide sequence
YGRKKRRQRRRKGTCY

SEQ ID NO: 87 - protein, 8 aa, Homo sapiens
GTCAGWMA

(19) United States
(12) Patent Application Publication
(10) Pub. No.: US 2020/0223894 A1
(43) Pub. Date: Jul. 16, 2020

(54) PEPTIDES FOR THE TREATMENT OF CONDITIONS ASSOCIATED WITH ADIPONECTIN DYSFUNCTION

(71) Applicant: AUCKLAND UNISERVICES LIMITED, Auckland (NZ)

(72) Inventors: Alok Mitra, Auckland (NZ); Mazdak Radjainia, Auckland (NZ); Lutz Hampe, Auckland (NZ)

(21) Appl. No.: 16/648,446

(22) PCT Filed: Sep. 19, 2018
(86) PCT No.: PCT/IB2018/057197

§ 371 Date: Mar. 18, 2020

(30) Foreign Application Priority Data
Sep. 19, 2017 (NZ) 735651

(51) Int. Cl. C07K 14/47

(52) U.S. Cl. CPC C07K 14/4703; A61K 38/00; C07K 2319/10

(57) ABSTRACT

The present invention provides peptides comprising a cargo peptide and a cell penetrating peptide that bind ERp44, peptides comprising a cargo peptide and a cell penetrating peptide that increase secretion of adiponectin from cells and methods of treating or preventing diseases associated with adiponectin dysfunction by administering said peptides.

### FIELD OF THE INVENTION

The present invention relates to peptides, pharmaceutical compositions comprising the peptides, methods of treating conditions associated with adiponectin dysfunction in a subject and manufacture of medicaments for the same.

### BACKGROUND TO THE INVENTION

Globally, obesity has assumed pandemic proportions. The number of overweight or obese individuals rose to about 2 billion in 2013 (Ng et al., 2014 Lancet, 384:766-781). Obesity-related diseases such as metabolic syndrome, insulin resistance, diabetes, musculoskeletal disease, cardiovascular disease, neurodegenerative disease and cancer cause significant morbidity and mortality. For example, the prevalence of diabetes has doubled worldwide since 1980 and the number of affected individuals is expected to rise to ˜590 million by 2035 (Global report on diabetes. World Health Organization, Geneva, 2016).

There is a continuing need for new therapies to treat or prevent obesity and obesity-related diseases.

It is an object of the present invention to provide peptides that meet this need, or to at least provide the public with a useful choice.

### SUMMARY OF THE INVENTION

In a first aspect the invention relates to a peptide comprising
one or more cargo peptides that bind ERp44, and
one or more cell penetrating agents;
   wherein the cargo peptide comprises or consists of the amino acid sequence L¹-X^{a}-C-X^{b}-L² , or a functional variant thereof, wherein
a) X^{a} and X^{b} are each independently 1 to 20 amino acids,
b) L¹ is a linker binding the cell penetrating agent and the cargo peptide, a linker binding the cargo peptide and another cargo peptide or L¹ is absent, and
c) L² is a linker binding the cell penetrating agent and the cargo peptide, a linker binding the cargo peptide and another cargo peptide or L² is absent,
   wherein at least one of L¹ and L² is present and is a linker binding the cell penetrating agent and the cargo peptide, and
   wherein X^{b} is not AGWMA or X^{b} is not AGWMA for at least one of the cargo peptides if the peptide comprises two or more cargo peptides.

In a second aspect the invention relates to a peptide comprising
one or more cargo peptides that bind ERp44, and
one or more cell penetrating agents;
   wherein the cargo peptide comprises or consists of the amino acid sequence L¹-X^{a}-C-X^{b}-L², or a functional variant thereof, wherein
a) X^{a} and X^{b} are each independently 1 to 20 amino acids,
b) L¹ is a linker binding the cell penetrating agent and the cargo peptide, a linker binding the cargo peptide and another cargo peptide or L¹ is absent, and
c) L² is a linker binding the cell penetrating agent and the cargo peptide, a linker binding the cargo peptide and another cargo peptide or L² is absent,
   wherein at least one of L¹ and L² is present and is a linker binding the cell penetrating agent and the cargo peptide, and
   wherein
(i) X^{b} is not AGWMA, or X^{b} is not AGWMA for at least one of the cargo peptides if the peptide comprises two or more cargo peptides, OR
(ii) X^{a} is not K, if X^{a} comprises 2 to 20 amino acids the N-terminal amino acid of X^{a} is not K, X^{a} is not K for at least one of the cargo peptides if the peptide comprises two or more cargo peptides, or if X^{a} comprises 2 to 20 amino acids the N-terminal amino acid of X^{a} is not K for at least one of the cargo peptides if the peptide comprises two or more cargo peptides.

In a third aspect the invention relates to a peptide comprising
one or more cargo peptides, and
one or more cell penetrating agents;
   wherein the cargo peptide comprises or consists of the amino acid sequence L¹-X^{a} -C-X^{b}-L², or a functional variant thereof, wherein
a) X^{a} and X^{b} are each 1 to 20 amino acids,
b) L¹ is a linker binding the cell penetrating agent and the cargo peptide, a linker binding the cargo peptide and another cargo peptide or L¹ is absent, and
c) L² is a linker binding the cell penetrating agent and the cargo peptide, a linker binding the cargo peptide and another cargo peptide or L² is absent,
   wherein at least one of L¹ and L² is present and is a linker binding the cell penetrating agent and the cargo peptide, and
   wherein the peptide induces an increase in total secreted adiponectin of at least about 10% as determined by incubating the peptide with 3T3-L1 adipocyte cells for 24 hours at a concentration of 200 nM and measuring total adiponectin in the culture medium of the cells compared to 3T3-L1 adipocyte cells incubated without the peptide under the same conditions.

In a fourth aspect the invention relates to a peptide comprising
one or more cargo peptides, and
one or more cell penetrating agents;
   wherein the cargo peptide comprises or consists of the amino acid sequence L¹ -X^{a}-C-X^{b}-L², or a functional variant thereof, wherein
d) X^{a} and X^{b} are each 1 to 20 amino acids,
e) L¹ is a linker binding the cell penetrating agent and the cargo peptide, a linker binding the cargo peptide and another cargo peptide or L¹ is absent, and
f) L² is a linker binding the cell penetrating agent and the cargo peptide, a linker binding the cargo peptide and another cargo peptide or L² is absent,
   wherein at least one of L¹ and L² is present and is a linker binding the cell penetrating agent and the cargo peptide, and
   wherein the peptide induces an increase in total secreted adiponectin of at least about 10% as determined by incubating the peptide with 3T3-L1 adipocyte cells for 24 hours at a concentration of 200 nM and measuring total adiponectin in the culture medium of the cells compared to 3T3-L1 adipocyte cells incubated with the cargo peptide under the same conditions.

In a fifth aspect the invention relates to a pharmaceutical composition comprising a peptide described herein and a pharmaceutically acceptable carrier.

In a sixth aspect the invention relates to a method of treating or preventing a condition associated with adiponectin dysfunction the method comprising administering to the subject a therapeutically effective amount of a peptide or pharmaceutical composition described herein.

In a seventh aspect the invention relates to use of a peptide or pharmaceutical composition described herein in the manufacture of a medicament for treating or preventing a disease associated with adiponectin dysfunction in a subject in need thereof.

In an eighth aspect the invention relates to a peptide or pharmaceutical composition described herein for treating or preventing a disease associated with adiponectin dysfunction in a subject in need thereof.

In a ninth aspect the invention relates to a method of treating or preventing a condition associated with adiponectin dysfunction in a subject in need thereof, the method comprising administering to the subject a gene therapy vector comprising a nucleotide sequence encoding a cargo peptide described herein wherein the vector induces expression of a therapeutically effective amount of the cargo peptide.

In a tenth aspect the invention relates to a gene therapy vector comprising a nucleotide sequence encoding a peptide described herein, said gene therapy vector being operably linked to an adipose tissue-specific promoter.

Any of the embodiments or preferences described herein may relate to any of the aspects herein alone or in combination with any one or more embodiments or preferences described herein, unless stated or indicated otherwise.

In various embodiments the peptide may induce an increase in total secreted adiponectin of at least about 10%, about 20%, about 25%, about 30%, about 40%, about 50%, about 60%, about 70%, about 75%, about 80%, about 90%, or at least about 100% as determined by incubating the peptide with 3T3-L1 adipocyte cells for 24 hours at a concentration of 200 nM and measuring total adiponectin in the culture medium of the cells compared to 3T3-L1 adipocyte cells incubated without the peptide under the same conditions.

In various embodiments the peptide may induce an increase in total secreted adiponectin of at least about 10%, about 20%, about 25%, about 30%, about 40%, about 50%, about 60%, about 70%, about 75%, about 80%, about 90%, or at least about 100% as determined by incubating the peptide with 3T3-L1 adipocyte cells for 24 hours at a concentration of 200 nM and measuring total adiponectin in the culture medium of the cells compared to 3T3-L1 adipocyte cells incubated with the cargo peptide under the same conditions.

In various embodiments the cargo peptide may comprise or consist of
a) three or more contiguous amino acids from the sequence

[SEQ ID No: 1]
AVDLGNIWRFPYICYQNGGGAF,

b) three or more contiguous amino acids from the sequence

[SEQ ID No: 2]
LISSFTDQLPWTSCKNSWNTGN,

c) three or more contiguous amino acids from the sequence

[SEQ ID No: 3]
PWTSCKNSWNTGNCTNYFAQDN,

d) three or more contiguous amino acids from the sequence

[SEQ ID No: 4]
QAFAQYTDKHGEVCPAGWKPGS,

e) three or more contiguous amino acids from the sequence

[SEQ ID No: 5]
YMPKKATELKHLQCLEEELKP,

f) three or more contiguous amino acids from the sequence

[SEQ ID No: 6]
IVLELKGSETTFMCEYADETAT,

g) three or more contiguous amino acids from the sequence

[SEQ ID No: 7]
ATIVEFLNRWITFCQSIISTLT,

h) three or more contiguous amino acids from the sequence RYCLL [SEQ ID No: 8],
i) three or more contiguous amino acids from the sequence

[SEQ ID No: 9]
DISQCGRRDCAVKPCQSDE,

j) the amino acid sequence YICYQ [SEQ ID No: 10],
k) the amino acid sequence TSCKN [SEQ ID No: 11],
l) the amino acid sequence GNCTN [SEQ ID No: 12],
m) the amino acid sequence HGEVCPAGW [SEQ ID No: 13],
n) the amino acid sequence EVCPA [SEQ ID No: 14],
o) the amino acid sequence LQCLE [SEQ ID No: 15],
p) the amino acid sequence FMCEY [SEQ ID No: 16],
q) the amino acid sequence TFCQS [SEQ ID No: 17],
r) the amino acid sequence SQCGRRDCAVKPCQS [SEQ ID No: 18],
s) three or more contiguous amino acids from the sequence PTLYNVSLVMSDTAGTCY [SEQ ID No: 19],
t) three or more contiguous amino acids from the sequence PTLYNVSLIMSDTGGTCY [SEQ ID No: 20],
u) three or more contiguous amino acids from the sequence

[SEQ ID No: 21]
PTHVNVSVVMAEVDGTCY,

v) three or more contiguous amino acids from the sequence

[SEQ ID No: 22]
PTNVSVVSVIMSEGDGICY,

w) the amino acid sequence DTAGTCY [SEQ ID No: 23],
x) the amino acid sequence DTGGTCY [SEQ ID No: 24],
y) the amino acid sequence GTCY [SEQ ID No: 25], or
z) a functional variant of any one of a) to y).

In various embodiments the cargo peptide may comprise or consist of
a) the amino acid sequence WTSCKNSW [SEQ ID No:73],
b) the amino acid sequence TGNCTNYF [SEQ ID No:75],
c) the amino acid sequence WTSCKNSWNTGNCTNY [SEQ ID No: 77],
d) the amino acid sequence GTCA [SEQ ID No: 83],
e) the amino acid sequence GTCYGWMA [SEQ ID No: 81], or
f) the amino acid sequence GTCAGWMA [SEQ ID No: 87] or
g) a functional variant of any one of a) to f).

In one embodiment the peptide may comprise
one or more cargo peptides, and
one or more cell penetrating agents;
   wherein the cargo peptide comprises or consists of the amino acid sequence L¹-X¹-X²-X³-C-X⁴-X⁵-L² or a functional variant thereof, wherein
a) X¹ is absent or is 1 to 20 amino acids,
b) X² is selected from the group comprising glycine, alanine, valine, leucine, isoleucine, methionine, proline, phenylalanine and tryptophan,
c) X³ is selected from the group comprising glycine, alanine, valine, leucine, isoleucine, methionine, proline, phenylalanine and tryptophan, serine, threonine, asparagine, glutamine, tyrosine, cysteine, lysine, arginine, histidine, aspartic acid and glutamic acid,
d) X⁴ is selected from the group comprising serine, threonine, asparagine, glutamine, tyrosine, cysteine, lysine, arginine, histidine, aspartic acid, glutamic acid, phenylalanine and tryptophan,
e) X⁵ is absent or is 1-20 amino acids,
f) L¹ comprises a linker binding the cell penetrating agent and the cargo peptide, a linker binding the cargo peptide and another cargo peptide or L¹ is absent, and
g) L² comprises a linker binding the cell penetrating agent and the cargo peptide, a linker binding the cargo peptide and another cargo peptide or L² is absent,
   wherein at least one of L¹ and L² is present and is a linker binding the cell penetrating agent and the cargo peptide.

In one embodiment the peptide may comprise
one or more cargo peptides, and
one or more cell penetrating agents;
   wherein the cargo peptide comprises or consists of the amino acid sequence L¹-X¹-X²-X³-C-X⁴-X⁵-L² or a functional variant thereof, wherein
a) X¹ is absent or is 1 to 20 amino acids,
b) X² is selected from the group comprising glycine, alanine, valine, leucine, isoleucine, methionine, proline, phenylalanine, threonine, serine and tryptophan,
c) X³ is selected from the group comprising glycine, alanine, valine, leucine, isoleucine, methionine, proline, phenylalanine and tryptophan, serine, threonine, asparagine, glutamine, tyrosine, cysteine, lysine, arginine, histidine, aspartic acid and glutamic acid,
d) X⁴ is selected from the group comprising serine, threonine, asparagine, glutamine, tyrosine, cysteine, lysine, arginine, histidine, aspartic acid, glutamic acid, phenylalanine, alanine, valine, glycine and tryptophan,
e) X⁵ is absent or is 1-20 amino acids,
f) L¹ comprises a linker binding the cell penetrating agent and the cargo peptide, a linker binding the cargo peptide and another cargo peptide or L¹ is absent, and
g) L² comprises a linker binding the cell penetrating agent and the cargo peptide, a linker binding the cargo peptide and another cargo peptide or L² is absent,
   wherein at least one of L¹ and L² is present and is a linker binding the cell penetrating agent and the cargo peptide.

In one embodiment X¹ is not lysine or if X¹ is 2 to 20 amino acids, the N-terminal amino acid of X¹ is not lysine. In another embodiment, if the peptide comprises two or more cargo peptides, X¹ is not lysine for at least one of the cargo peptides or if X¹ is 2 to 20 amino acids, the N-terminal amino acid of X¹ is not lysine for at least one of the cargo peptides.

In various embodiments X² may be selected from the group comprising
a) glycine, alanine, valine and leucine, and
b) glycine and alanine.

In various embodiments X² may be selected from the group comprising
a) glycine, alanine, valine, threonine, serine and leucine,
b) glycine, threonine, serine and alanine, and
c) glycine or threonine.

In various embodiments X³ may be selected from the group comprising
a) serine, threonine, asparagine, glutamine, tyrosine, cysteine, lysine, arginine, histidine, aspartic acid and glutamic acid,
b) serine, threonine, asparagine, glutamine, cysteine and tyrosine,
c) serine, threonine, asparagine, and glutamine,
d) threonine, serine and tyrosine, and
e) threonine and serine.

In various embodiments X⁴ may be selected from the group comprising
a) serine, threonine, asparagine, glutamine, tyrosine, cysteine, lysine, arginine, histidine, aspartic acid and glutamic acid,
b) serine, threonine, asparagine, glutamine, tyrosine and cysteine, and
c) tyrosine, phenylalanine and tryptophan.

In various embodiments X⁴ may be selected from the group comprising
a) serine, threonine, asparagine, glutamine, tyrosine, cysteine, lysine, arginine, histidine, aspartic acid, alanine, glycine, valine, and glutamic acid,
b) serine, threonine, asparagine, glutamine, tyrosine, alanine, valine, glycine and cysteine,
c) tyrosine, phenylalanine, alanine, glycine, valine, and tryptophan, and
d) tyrosine and alanine.

In one embodiment
a) X² may be selected from the group comprising
   i. tyrosine, phenylalanine, tryptophan, threonine, serine, asparagine, glutamine, cysteine, glycine, alanine, valine, leucine, isoleucine, methionine, and proline,
   ii. tyrosine, phenylalanine, tryptophan, threonine, serine, glycine, alanine, valine and leucine, and
   iii. tyrosine, threonine and glycine,
b) X³ may be selected from the group comprising
   i. serine, threonine, asparagine, glutamine, glycine, tyrosine, cysteine, isoleucine, valine, leucine, alanine, proline, phenylalanine, methionine and tryptophan,
   ii. serine, asparagine, threonine, asparagine, glutamine, isoleucine, leucine and valine, and
   iii. serine, asparagine, and isoleucine,
c) X⁴ may be selected from the group comprising
   i. tyrosine, phenylalanine, tryptophan, lysine, arginine, histidine, threonine, serine, asparagine, cysteine and glutamine,
   ii. tyrosine, phenylalanine, tryptophan, lysine, arginine, threonine and serine, and
   iii. tyrosine, lysine and threonine, and
d) X⁵ may be selected from the group comprising
   i. 1 to 12 amino acids,
   ii. 1 to 12 amino acids with asparagine, glutamine, serine, cysteine or threonine as the N-terminal amino acid, and
   iii. 1 to 12 amino acids with asparagine or glutamine as the N-terminal amino acid, and
   iv. asparagine and glutamine.

In one embodiment the peptide may comprise
one or more cargo peptides, and
one or more cell penetrating agents;
   wherein the cargo peptide comprises or consists of the amino acid sequence L¹-X¹-X²-X³-C-X⁴-X⁵-L² or a functional variant thereof, wherein
a) X¹ is absent or is 1 to 20 amino acids,
b) X² is any amino acid,
c) X³ is selected from the group comprising serine, asparagine, isoleucine, valine, glutamine, methionine, phenylalanine, tyrosine, aspartic acid, threonine, asparagine, glutamine, cysteine, isoleucine, valine, leucine, alanine, proline, phenylalanine, methionine, tryptophan, histidine and glutamic acid,
d) X⁴ is any amino acid,
e) X⁵ is absent or is 1-20 amino acids,
f) L¹ comprises a linker binding the cell penetrating agent and the cargo peptide, a linker binding the cargo peptide and another cargo peptide or L¹ is absent, and
g) L² comprises a linker binding the cell penetrating agent and the cargo peptide, a linker binding the cargo peptide and another cargo peptide or L² is absent,
   wherein at least one of L¹ and L² is present and is a linker binding the cell penetrating agent and the cargo peptide.

In various embodiments, for example, if the cargo peptide is derived from a SERT ERp44 binding domain,
a) X² may be selected from the group comprising
   i. tyrosine, phenylalanine, tryptophan, threonine, serine, asparagine, glutamine, cysteine, glycine, alanine, valine, leucine, isoleucine, methionine, and proline,
   ii. tyrosine, phenylalanine, tryptophan, threonine, serine, glycine, alanine, valine and leucine, and
   iii. tyrosine, threonine and glycine,
b) X³ may be selected from the group comprising
   i. serine, threonine, asparagine, glutamine, glycine, tyrosine, cysteine, isoleucine, valine, leucine, alanine, proline, phenylalanine, methionine and tryptophan,
   ii. serine, asparagine, threonine, asparagine, glutamine, isoleucine, leucine and valine, and
   iii. serine, asparagine, and isoleucine,
c) X⁴ may be selected from the group comprising
   i. tyrosine, phenylalanine, tryptophan, lysine, arginine, histidine, threonine, serine, asparagine, cysteine and glutamine,
   ii. tyrosine, phenylalanine, tryptophan, lysine, arginine, threonine and serine, and
   iii. tyrosine, lysine and threonine, and
d) X⁵ may be selected from the group comprising
   i. 1 to 12 amino acids,
   ii. 1 to 12 amino acids with asparagine, glutamine, serine, cysteine or threonine as the N-terminal amino acid,
   iii. 1 to 12 amino acids with asparagine or glutamine as the N-terminal amino acid, and
   iv. asparagine and glutamine.

In one embodiment
a) X² may be selected from tyrosine, phenylalanine, tryptophan, and histidine,
b) X³ may be selected from isoleucine, valine, leucine, alanine, proline, phenylalanine, methionine and tryptophan,
c) X⁴ may be selected from tyrosine, phenylalanine, tryptophan, histidine, and
d) X⁵ may be asparagine or glutamine.

In one embodiment
a) X² may be selected from threonine, serine, asparagine, glutamine and cysteine,
b) X³ may be selected from the group comprising serine, threonine, asparagine, glutamine, glycine, tyrosine and cysteine
c) X⁴ may be selected from lysine, arginine and histidine, and
d) X⁵ may be selected from
   i. 1 to 12 amino acids,
   ii. 1 to 12 amino acids with asparagine, glutamine, serine, cysteine or threonine as the N-terminal amino acid,
   iii. 1 to 12 amino acids with asparagine or glutamine as the N-terminal amino acid, and
   iv. asparagine and glutamine.

In one embodiment
a) X² may be selected from glycine, phenylalanine, alanine, valine, leucine, isoleucine, methionine, tryptophan, and proline,
b) X³ may be selected from serine, threonine, asparagine, glutamine, glycine, tyrosine, and cysteine,
c) X⁴ may be selected from threonine, serine, asparagine, cysteine tyrosine, and glutamine, and
d) X⁵ may be selected from
   i. 1 to 12 amino acids,
   ii. 1 to 12 amino acids with asparagine, glutamine, serine, cysteine or threonine as the N-terminal amino acid,
   iii. 1 to 12 amino acids with asparagine or glutamine as the N-terminal amino acid, and
   iv. asparagine and glutamine.

In various embodiments, for example, if the cargo peptide is derived from a PRX4 ERp44 binding domain,
a) X² may be selected from glutamic acid and aspartic acid,
b) X³ may be selected from
   i. valine, alanine, leucine, isoleucine, proline, phenylalanine, methionine, tryptophan, and
   ii. valine, leucine or isoleucine,
c) X⁴ may be selected from
   i. proline, alanine, valine, leucine, isoleucine, phenylalanine, methionine and tryptophan, and
   ii. proline, and
d) X⁵ may be selected from the group comprising
   i. 1 to 10 amino acids with alanine, valine, leucine, isoleucine, proline, phenylalanine, methionine or tryptophan as the N-terminal amino acid,
   ii. 1 to 10 amino acids with alanine as the N-terminal amino acid, and
   iii. alanine.

In various embodiments, for example, if the cargo peptide is derived from an IL-2 ERp44 binding domain,
a) X² may be selected from the group comprising
   i. phenylalanine, tyrosine, tryptophan, histidine, threonine, serine, asparagine, glutamine, cysteine, leucine, glycine, alanine, valine, isoleucine, methionine, and proline,
   ii. phenylalanine, tyrosine, tryptophan, threonine, serine, leucine, valine, isoleucine, and,
   iii. phenylalanine, threonine or leucine,
b) X³ may be selected from the group comprising
   i. glutamine, asparagine, serine, threonine, glycine, tyrosine, cysteine, phenylalanine, tryptophan, histidine, methionine, isoleucine, valine, leucine, alanine, proline and glycine,
   ii. glutamine, asparagine, phenylalanine, tyrosine, tryptophan and methionine, and
   iii. glutamine, phenylalanine and methionine,
c) X⁴ may be selected from the group comprising
   i. leucine, alanine, valine, isoleucine, methionine, phenylalanine, proline, tryptophan, glutamic acid, aspartic acid, glutamine, asparagine, serine, threonine, glycine, tyrosine and cysteine,
   ii. leucine, valine, isoleucine, glutamic acid, aspartic acid, glutamine and asparagine, and
   iii. leucine, glutamic acid and glutamine, and
d) X⁵ may be selected from the group comprising
   i. glutamic acid, aspartic acid, tyrosine, phenylalanine, tryptophan, serine, threonine, glycine, asparagine, glutamine and cysteine,
   ii. glutamic acid, aspartic acid, tyrosine, phenylalanine, serine and threonine, and
   iii. glutamic acid, tyrosine, and serine.

In one embodiment
a) X² may be selected from the group comprising
   i. leucine, glycine, alanine, valine, isoleucine, methionine, and proline, and ii. leucine, valine, and isoleucine,
b) X³ may be selected from the group comprising
   i. glutamine, asparagine, serine, threonine, glycine, tyrosine and cysteine, and
   ii. glutamine and asparagine,
c) X⁴ may be selected from the group comprising
   i. leucine, alanine, valine, isoleucine, methionine, phenylalanine, proline, and tryptophan, and
   ii. leucine, valine and isoleucine, and
d) X⁵ may be glutamic acid or aspartic acid.

In one embodiment
a) X² may be selected from phenylalanine, tyrosine, tryptophan and histidine,
b) X³ may be selected from
   i. phenylalanine, tryptophan, methionine, isoleucine, valine, leucine, alanine, proline and glycine, and
   ii. methionine,
c) X⁴ may be glutamic acid or aspartic acid, and
d) X⁵ may be selected from tyrosine, phenylalanine and tryptophan.

In one embodiment
a) X² may be selected from the group comprising
   i. threonine, serine, asparagine, glutamine, cysteine, and glycine,
   ii. threonine and serine,
b) X³ may be selected from the group comprising
   i. tyrosine, phenylalanine, tryptophan, histidine, methionine, isoleucine, valine, leucine, alanine, proline and glycine, and
   ii. phenylalanine, tyrosine and tryptophan,
c) X⁴ may be selected from the group comprising
   i. glutamine, asparagine, serine, threonine, glycine, tyrosine, and cysteine, and
   ii. glutamine and asparagine, and
d) X⁵ may be selected from the group comprising
   i. serine, threonine, glycine, asparagine, glutamine, and cysteine, and
   ii. serine and threonine.

In one embodiment
a) X² may be selected from arginine, lysine and histidine,
b) X³ may be selected from the group comprising
   i. tyrosine, phenylalanine, tryptophan, histidine, methionine, isoleucine, valine, leucine, alanine, proline and glycine, and
   ii. phenylalanine, tyrosine or tryptophan, and
c) X⁴ and X⁵ may each independently be selected from the group comprising
   i. leucine, alanine, valine, isoleucine, proline, phenylalanine, methionine and tryptophan, and
   ii. leucine and isoleucine.

In one embodiment
a) X² may be selected from the group comprising
   i. serine, threonine, arginine, glycine, asparagine, glutamine, tyrosine, cysteine lysine, arginine and histidine, and
   ii. serine, threonine, arginine and lysine,
b) X³ may be selected from the group comprising
   i. glutamine, glycine, asparagine, serine, threonine, tyrosine, cysteine, aspartic acid, glutamic acid, proline, alanine, valine, leucine, isoleucine, phenylalanine, methionine and tryptophan,
   ii. glutamine, asparagine, aspartic acid, glutamic acid and proline, and
   iii. glutamine, aspartic acid and proline,
c) X⁴ may be selected from the group comprising
   i. glycine, asparagine, glutamine, serine, threonine, tyrosine, cysteine, alanine, valine, leucine, isoleucine, proline, phenylalanine, methionine, tryptophan and glutamine,
   ii. glycine, alanine, glutamine and asparagine, and
   iii. glycine, alanine, and glutamine, and
d) X⁵ may be 1-15 amino acids.

In one embodiment the peptide may be a cyclic peptide.

In various embodiments the peptide may comprise 1, 2, 3, 4, 5, 6, 7, 8, 9 or 10 or more cargo peptides, and ranges may be selected from between any of these values, for example, from about 1 to about 10, 1 to about 7, 1 to about 5, 1 to about 3, 2 to about 10, 2 to about 5, or about 2 to about 3 cargo peptides.

In one embodiment the peptide may bind ERp44 at a pH of about 6.5 as determined by incubating ERp44 with a 10 fold excess of the peptide for one week at pH 6.5 and detecting ERp44-peptide interaction by electrospray ionization mass spectrometry.

In one embodiment the peptide may bind ERp44 at a pH of about 6.5 and release ERp44 at a pH of about 8.

In one embodiment the amount of a peptide-ERp44 complex present at pH 8 is less than about 90% of the amount of peptide-ERp44 complex present at pH 6.5 as determined by incubating the peptide with ERp44 at a ratio of 10:1 at pH 6.5 and increasing the pH to 8.0 and comparing the degree of ERp44-peptide interaction at pH 6.5 and pH 8.0.

In various embodiments X¹, X⁵, X^{a} and X^{b} may be each independently 1, 2, 3, 4, 5, 6, 7, 8, 9, 10, 11, 12, 13, 14, 15, 16, 17, 18, 19 or 20 amino acids, or a range selected from between any of these values, for example from 1 to 2, 1 to 3, 1 to 4, 1 to 5, 1 to 6, 1 to 7, 1 to 8, 1 to 9, 1 to 10, 1 to 11, 1 to 12, 1 to 13, 1 to 14, 1 to 15, 1 to 16, 1 to 17, 1 to 18, 1 to 19, 1 to 20, 2 to 3, 2 to 4, 2 to 5, 2 to 6, 2 to 10, 3 to 4, 3 to 5, 3 to 6, 3 to 10, 4 to 5, 4 to 6, 4 to 10, 4 to 6, 5 to 10 or 6 to 10 amino acids.

In various embodiments the linker may comprise a peptide bond, one or more amino acids, a covalent bond, or a chemical spacer.

In various embodiments the cell penetrating agent may comprise a cell penetrating peptide.

In various embodiments the cell penetrating peptide may be selected from the group comprising a cationic cell penetrating peptide, an amphipathic cell penetrating peptide and a hydrophobic cell penetrating peptide.

In various embodiments the cell penetrating peptide may be selected from the group consisting of TAT, Penetratin (Antp), DPV1047, Bac, SynB1, SynB1-NLS, Poly-arginine, VP22, Transportan, MAP, pVEC, MTS, hCT derived, MPG, Buforin 2, PEP-1, Magainin 2, M918 C105Y, PFVYLI, BPrPR(1-28), ARF(1-22), p28, VT5, YTA2, YTA4, CADY and PEP-7.

In one embodiment the cell penetrating agent may be a cell penetrating peptoid. In one embodiment the cell penetrating peptoid may be an oligomer of N-substituted glycine units.

In various embodiments the pharmaceutical composition may be formulated for oral or parenteral administration. In one embodiment the pharmaceutical composition may be formulated for oral administration and may be a delayed release dosage form. In one embodiment the pharmaceutical composition may be formulated for oral administration and may comprise an enteric dosage form including an enterically coated dosage form. In one embodiment the pharmaceutical composition may be formulated to be injected into the subject.

In various embodiments the condition associated with adiponectin dysfunction may be a condition associated with reduced serum adiponectin, reduced insulin sensitivity, increased serum cholesterol, increased serum triglycerides, or increased blood glucose.

In various embodiments the condition associated with adiponectin dysfunction may be metabolic syndrome, insulin resistance, diabetes, musculoskeletal disease, musculoskeletal disease, cardiovascular disease, respiratory disease, gallbladder disease, liver disease, gynaecological disease, sexual dysfunction, neurodegenerative disease or cancer.

In various embodiments the condition associated with adiponectin dysfunction may be type 2 diabetes, hypertension, hypercholesterolemia, hypertriglyceridemia, osteoarthritis, atherosclerosis, heart attack, stroke, sleep apnoea, nonalcoholic fatty liver disease, infertility, irregular menstruation, erectile dysfunction, Alzheimer's disease,

Parkinson's disease, endometrial cancer, uterine cancer, breast cancer, ovarian cancer, prostate cancer, liver cancer, gallbladder cancer, kidney cancer, rectal cancer, oesophageal cancer, gallbladder cancer or colon cancer.

In one embodiment the nucleotide sequence encoding the cargo peptide may be under the control of one or more regulatory sequences that induce expression of the cargo peptide in a specific cell type or tissue. In one embodiment the regulatory sequence may induce expression of the cargo peptide in adipocytes or adipose tissue.

In various embodiments the nucleotide sequence may encode a cargo peptide comprising or consisting of
a) three or more contiguous amino acids from the sequence

[SEQ ID No: 1]
AVDLGNIWRFPYICYQNGGGAF,

b) three or more contiguous amino acids from the sequence

[SEQ ID No: 2]
LISSFTDQLPWTSCKNSWNTGN,

c) three or more contiguous amino acids from the sequence

[SEQ ID No: 3]
PWTSCKNSWNTGNCTNYFAQDN,

d) three or more contiguous amino acids from the sequence

[SEQ ID No: 4]
QAFAQYTDKHGEVCPAGWKPGS,

e) three or more contiguous amino acids from the sequence

[SEQ ID No: 5]
YMPKKATELKHLQCLEEELKP,

f) three or more contiguous amino acids from the sequence

[SEQ ID No: 6]
IVLELKGSETTFMCEYADETAT,

g) three or more contiguous amino acids from the sequence

[SEQ ID No: 7]
ATIVEFLNRWITFCQSIISTLT,

h) three or more contiguous amino acids from the sequence RYCLL [SEQ ID No: 8],
i) three or more contiguous amino acids from the sequence

[SEQ ID No: 9]
DISQCGRRDCAVKPCQSDE,

j) the amino acid sequence YICYQ [SEQ ID No: 10],
k) the amino acid sequence TSCKN [SEQ ID No: 11],
l) the amino acid sequence GNCTN [SEQ ID No: 12],
m) the amino acid sequence HGEVCPAGW [SEQ ID No: 13],
n) the amino acid sequence EVCPA [SEQ ID No: 14],
o) the amino acid sequence LQCLE [SEQ ID No: 15],
p) the amino acid sequence FMCEY [SEQ ID No: 16],
q) the amino acid sequence TFCQS [SEQ ID No: 17],
r) the amino acid sequence SQCGRRDCAVKPCQS [SEQ ID No: 18],
s) three or more contiguous amino acids from the sequence PTLYNVSLVMSDTAGTCY [SEQ ID No: 19],
t) three or more contiguous amino acids from the sequence PTLYNVSLIMSDTGGTCY [SEQ ID No: 20],
u) three or more contiguous amino acids from the sequence

[SEQ ID No: 21]
PTHVNVSVVMAEVDGTCY,

v) three or more contiguous amino acids from the sequence

[SEQ ID No: 22]
PTNVSVVSVIMSEGDGICY,

w) the amino acid sequence DTAGTCY [SEQ ID No: 23],
x) the amino acid sequence DTGGTCY [SEQ ID No: 24], and
y) the amino acid sequence GTCY [SEQ ID No: 25].

In various embodiments the nucleotide sequence may encode a cargo peptide comprising or consisting of
a) the amino acid sequence WTSCKNSW [SEQ ID No:73],
b) the amino acid sequence TGNCTNYF [SEQ ID No:75],
c) the amino acid sequence WTSCKNSWNTGNCTNY [SEQ ID No: 77],
d) the amino acid sequence GTCA [SEQ ID No: 83],
e) the amino acid sequence GTCYGWMA [SEQ ID No: 81], or
f) the amino acid sequence GTCAGWMA [SEQ ID No: 87].

The term “comprising” as used in this specification means “consisting at least in part of”. When interpreting statements in this specification which include that term, the features, prefaced by that term in each statement, all need to be present but other features can also be present. Related terms such as “comprise” and “comprised” are to be interpreted in the same manner.

This invention may also be said broadly to consist in the parts, elements and features referred to or indicated in the specification of the application, individually or collectively, and any or all combinations of any two or more of said parts, elements or features, and where specific integers are mentioned herein which have known equivalents in the art to which this invention relates, such known equivalents are deemed to be incorporated herein as if individually set forth.

It is intended that reference to a range of numbers disclosed herein (for example, 1 to 10) also incorporates reference to all rational numbers within that range (for example, 1, 1.1, 2, 3, 3.9, 4, 5, 6, 6.5, 7, 8, 9, and 10) and also any range of rational numbers within that range (for example, 2 to 8, 1.5 to 5.5, and 3.1 to 4.7) and, therefore, all sub-ranges of all ranges expressly disclosed herein are hereby expressly disclosed. These are only examples of what is specifically intended and all possible combinations of numerical values between the lowest value and the highest value enumerated are to be considered to be expressly stated in this application in a similar manner.

In this specification, where reference has been made to external sources of information, including patent specifications and other documents, this is generally for the purpose of providing a context for discussing the features of the present invention. Unless stated otherwise, reference to such sources of information is not to be construed, in any jurisdiction, as an admission that such sources of information are prior art or form part of the common general knowledge in the art.

### BRIEF DESCRIPTION OF THE DRAWINGS

The invention will now be described by way of example only and with reference to the drawings in which:

FIG. 1 is a series of bar graphs showing adiponectin levels in the cell lysate and conditioned media of murine 3T3-L1 adipocytes treated with peptides of the invention (200 nM). A) shows adiponectin levels in the media (left panel) and cell lysate (right panel) of cells treated with IgM-derived peptides M1 (1) and M2 (2). B) shows adiponectin levels in the media (left panel) and cell lysate (right panel) of cells treated with an adiponectin-derived peptide. Results are shown for each cargo peptide alone (white bars) and each CPP-cargo peptide (checked bars). *, P<0.05 compared with the corresponding non-CPP cargo peptide (n=6).

FIG. 2 is a series of bar graphs showing the oligomeric distribution of adiponectin in the cell lysate and conditioned media of murine 3T3-L1 adipocytes treated with peptides of the invention (200 nM). Each graph shows the distribution of adiponectin trimers (a), hexamers (b) and HMW adiponectin (c). A) shows the oligomeric distribution of adiponectin in the conditioned media of cells treated with IgM-derived peptides M1 (left panel) and M2 (right panel). B) shows the oligomeric distribution of adiponectin in the conditioned media of cells treated with an adiponectin-derived peptide. C) shows the oligomeric distribution of adiponectin in the cell lysate of cells treated with IgM-derived peptides M1 (left panel) and M2 (right panel). D) shows the oligomeric distribution of adiponectin in the cell lysate of cells treated with an adiponectin-derived peptide. Results are shown for each cargo peptide alone (white bars) and each CPP-cargo peptide (checked bars). *, P<0.05 compared with the corresponding non-CPP cargo peptide (n=6).

FIG. 3 is a bar graph showing ERp44-adiponectin interactions in adipocytes treated with peptides of the invention. Co-immunoprecipitation was performed to determine ERp44-adiponectin interactions in the cell lysate of 3T3-L1 adipocytes treated with 1) an adiponectin-derived peptide or IgM-derived peptides 2) M1 and 3) M2. Results are shown for each cargo peptide alone (white bars) and each CPP-cargo peptide (checked bars). *, P<0.05 compared with the corresponding non-CPP cargo peptide groups (n=6).

FIG. 4 is a series of bar graphs showing total and HMW adiponectin levels and ERp44-adiponectin interactions in C57/BL6 mice fed a high fat diet for eight weeks and treated with peptides of the invention. A) shows serum adiponectin levels in mice treated with IgM-derived peptides M1 (left panel), M2 (centre panel) or an adiponectin-derived peptide (right panel) (10 mg/kg, intraperitoneal administration). (B) shows serum HMW adiponectin in mice treated with IgM-derived peptides M1 (left panel) and M2 (right panel) normalized to the corresponding cargo peptides at time zero and presented as fold change. C) shows serum HMW adiponectin in mice treated with an adiponectin-derived peptide normalized to the corresponding cargo peptide at time zero and presented as fold change. (D) shows ERp44-adiponectin interaction in adipose tissue lysates of mice treated with (1) adiponectin-derived peptides, and IgM-derived peptides M1 (2) and M2 (3). Results are shown for each cargo peptide alone (white bars) and each CPP-cargo peptide (checked bars). *, P<0.05 compared with the corresponding non-CPP cargo peptide groups (n=6).

FIG. 5 is a series of bar graphs showing fasting body weight and body fat mass in C57/BL6 mice fed a standard chow diet (STC) or high fat diet (HFD) for eight weeks and treated with peptides of the invention. A) shows fasting body weight and B) shows total body fat mass in mice treated with M1 (left panel) and M2 peptides (right panel) (5 mg/kg/day i.p.) for four weeks. Results are shown for mice on STC diet and administered cargo peptide (white bars) or CPP-peptide (checked bars) and mice on HFD diet and administered cargo peptide (bars with horizontal lines) or CPP-peptide (bars with vertical lines). *, P<0.05 compared with the corresponding non-CPP cargo peptide groups (n=6).

FIG. 6 is a series of bar graphs showing fasting blood glucose and insulin sensitivity in C57/BL6 mice treated with peptides of the invention. A) shows fasting blood glucose and B) shows insulin sensitivity in mice fed (1) a standard chow diet (STC) or (2) a high fat diet (HFD) for eight weeks then treated with M1 (white bars), CPP-M1 (checked bars), M2 (bars with horizontal lines) and CPP-M2 peptides (vertical lines) (5 mg/kg/day i.p.) for four weeks. *, P<0.05 compared with the corresponding non-CPP cargo peptide groups (n=6).

FIG. 7 is a series of bar graphs showing serum adiponectin levels, serum triglycerides, circulating cholesterol and liver triglycerides in C57/BL6 mice treated with peptides of the invention. A) shows serum adiponectin, B) shows serum triglycerides; C) shows circulating cholesterol; and D) shows liver triglycerides in mice fed (1) a STC diet or (2) HFD and treated for four weeks with M1 (white bars), CPP-M1 (checked bars), M2 (bars with horizontal lines) and CPP-M2 peptides (vertical lines) (5 mg/kg/day i.p.). *, P<0.05 compared with the corresponding non-CPP cargo peptide groups (n=6).

FIG. 8 is a series of mass spectra showing formation of disulfide-bonded complexes between ERp44 and peptides of the invention. Recombinant mouse ERp44 (43 μM) was incubated with a 10-fold excess (430 μM) of A) an adiponectin derived peptide, B) CPP-adiponectin peptide, and IgM-derived peptides C) M1, D) CPP-M1, E) M2 and F) CPP-M2 for 4 days and the mixture was subsequently analyzed by ESI-MS to quantify the amount of disulfide linked complex formed. Each of the spectra contains two sets of peaks, one at 46,074 atomic mass units (amu) representing free ERp44 (1) and a second representing the disulfide-linked ERp44-peptide complex (2).

FIG. 9 is a bar graph showing adiponectin levels in the conditioned media of murine 3T3-L1 adipocytes at 8 hours (white bars) and 24 hours (black bars) after treatment with peptides of the invention (200 nM). Fold change in adiponectin was calculated relative to time zero samples.

FIG. 10 is a line graph showing adiponectin levels measured in serum samples taken over 48 hours from C57/BL6 mice fed a high fat diet for eight weeks and injected with the following peptides of the invention (10 mg/kg, intraperitoneal administration): CPP-M1 (circles); S16 (squares); 148-1 (triangles) and 152-1 (inverted triangles). Fold change in adiponectin was calculated relative to time zero samples.

FIG. 11 is a bar graph showing relative production of adiponectin in C57/BL6 mice fed a high fat diet for eight weeks and injected with peptides of the invention (10 mg/kg, intraperitoneal administration). For each peptide, fold change in serum adiponectin was determined 1, 4, 8, 24 and 48 hours after injection relative to serum adiponectin at time zero and a curve prepared like those shown in FIG. 10. The area under the curve calculated for each peptide is shown in FIG. 11.

FIG. 12 is a series of bar graphs showing the relative levels of HMW adiponectin and adiponectin hexamer in serum from C57/BL6 mice fed a high fat diet for eight weeks and treated with peptides of the invention. SDS-PAGE was performed for serum samples taken over a time course of 48 hours after injection of A) 148-1 or B) 152-1. Western blotting was performed to detect HMW adiponectin and adiponectin hexamers. The intensity of protein bands corresponding to each oligomer were quantified using ImageJ and a ratio indicating the relative amount of HMW to hexamer was calculated.

### DETAILED DESCRIPTION OF THE INVENTION

The present inventors have surprisingly determined that peptides that bind the molecular chaperone ERp44 that are not derived from adiponectin may be used to treat or prevent obesity and conditions associated with adiponectin dysfunction. Thus the present invention relates to peptides comprising one or more cargo peptides that bind ERp44 and a cell penetrating agent, pharmaceutical compositions comprising the peptides and methods for the treatment or prevention of conditions associated with adiponectin dysfunction by administration of the peptides to a subject in need thereof.

### 1. Definitions

The term “amino acid” refers to a molecule containing both an amino group and a carboxyl group bound to a carbon which is designated the α-carbon. Suitable amino acids include, without limitation, both the D- and L-isomers of the naturally-occurring amino acids, as well as non-naturally occurring amino acids prepared by organic synthesis or other metabolic routes. Unless the context specifically indicates otherwise, the term amino acid, as used herein, is intended to include amino acid analogs.

In certain embodiments the peptide may comprise only natural amino acids. The term “naturally occurring amino acid” refers to any one of the twenty amino acids commonly found in peptides synthesized in nature, and known by the one letter abbreviations A, R, N, C, D, Q, E, G, H, I, L, K, M, F, P, S, T, W, Y and V.

The term “amino acid analog” or “non-naturally occurring amino acid” refers to a molecule which is structurally similar to an amino acid and which can be substituted for an amino acid. Examples of amino acid analogs include, but are not limited to, phosphorylated or glycosylated amino acids, N-alkylated amino acids (e.g. N-methyl amino acids), D-amino acids, β-amino acids, and γ-amino acids.

The term “conservative substitution” as used herein, refers to the replacement of an amino acid present in the native sequence of a peptide with a naturally or non-naturally occurring amino acid having a chemically similar or derivatised side chain, or a similarity in polarity, charge, solubility, hydrophobicity, hydrophilicity and/or the amphipathic nature to the amino acid present in the native sequence. Families of amino acid residues having similar side chains, for example, have been defined in the art. These families include, for example, amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). In certain embodiments, the hydrophobic index of amino acids may be considered in choosing suitable mutations. The importance of the hydrophobic amino acid index in conferring interactive biological function on a polypeptide is generally understood in the art. Alternatively, the substitution of like amino acids can be made effectively on the basis of hydrophilicity. The importance of hydrophilicity in conferring interactive biological function of a polypeptide is generally understood in the art. The use of the hydrophobic index or hydrophilicity in designing polypeptides is further discussed in U.S. Pat. No. 5,691,198.

An “effective amount” is an amount sufficient to effect beneficial or desired results including clinical results. An effective amount can be administered in one or more administrations by various routes of administration.

The effective amount will vary depending on, among other factors, the disease indicated, the severity of the disease, the age and relative health of the subject, the potency of the peptide administered, the mode of administration and the treatment desired. A person skilled in the art will be able to determine appropriate dosages having regard to these any other relevant factors.

The term “functional variant” is used herein to refer to a cargo peptide that binds the molecular chaperone ERp44, induces an increase in secreted adiponectin from adipocytes and that can be used to treat or prevent a condition associated with adiponectin dysfunction. Potential functional variants may be tested for these properties according to methods described herein, including in the Examples. The functional variant may comprise or consist of a naturally occurring (an allelic variant, for example) or non-naturally occurring (an artificially generated mutant, for example) peptide that varies from the amino acid sequence of a cargo peptide described herein by the addition, deletion or substitution of one or more amino acids.

In various embodiments the functional variant may comprise or consist of an amino acid sequence having at least about 70%, 75%, 80%, 85%, 90%, 95% or 99% sequence identity to the amino acid sequence of a cargo peptide sequence described herein.

The term “gene therapy vector” as used herein refers to a nucleic acid vector suitable for delivering a nucleotide sequence encoding a cargo peptide to a cell or tissue and providing for expression of the cargo peptide in that cell or tissue. Any suitable nucleic acid vector may be used, for example, a viral vector such as an adenoviral vector or a retroviral vector, or a non viral vector.

Body mass index (BMI) is a simple index that is frequently used to classify overweight and obesity. A subject's BMI is determined by dividing the subject's weight (in kilograms) by the square of the subject's height (in metres).

As used herein the term “obesity” or “obese” is used to describe a subject suffering from abnormal or excessive fat accumulation that presents a risk to health. An adult subject with a BMI of 30 or more is obese. A child subject aged between 5 and 19 years with a BMI greater than 2 standard deviations above the WHO growth reference median (available: http://www.who.int/growthref/who2007_bmi_for_age/en/) is considered obese.

As used herein the term “overweight” is used to describe an adult subject with a BMI of more than 25, or a child subject aged between 5 and 19 years with a BMI greater than 1 standard deviation above the WHO growth reference median.

The term “peptide” and the like is used herein to refer to any polymer of amino acid residues of any length. The polymer can be linear or non-linear (e.g., branched or cyclic), it can comprise modified amino acids or amino acid analogs. The term also encompasses amino acid polymers that have been modified naturally or by intervention, for example, by disulfide bond formation, glycosylation, lipidation, acetylation, phosphorylation, or any other modification or manipulation, for example conjugation with labeling or bioactive components.

The term “pharmaceutically acceptable carrier” refers to a carrier (adjuvant or vehicle) that may be administered to a subject together with the peptide described herein, or a pharmaceutically acceptable salt or solvate thereof.

A “subject” refers to a vertebrate that is a mammal, for example, a human. Mammals include, but are not limited to, humans, farm animals, sport animals, pets, primates, mice and rats.

The term “treatment”, and related terms such as “treating” and “treat”, as used herein relates generally to treatment, of a human or a non-human subject, in which some desired therapeutic effect is achieved. The therapeutic effect may, for example, be inhibition, reduction, amelioration, halt, or prevention of a disease or condition.

### 2. Peptides

The peptides of the invention comprise one or more cargo peptides and one or more cell penetrating agents.

Cargo peptide

The present inventors have determined that, surprisingly, peptides comprising a cell penetrating agent and a cargo peptide that binds ERp44 increase secretion of adiponectin, particularly high molecular weight adiponectin, from adipocytes and can be used to treat or prevent conditions associated with adiponectin dysfunction.

Adiponectin is a serum protein derived from adipocytes (fat cells). Adiponectin plays a protective role against obesity-related diseases. The beneficial effects of adiponectin such as insulin sensitisation are attributed mostly to the high molecular weight (HMW) adiponectin isoform.

The serum level of adiponectin is controlled by chaperones, principally ERp44, which sequester adiponectin in the endoplasmic reticulum (ER). Without wishing to be bound by any theory, it is believed that the peptides of the invention bind ERp44 and inhibit ERp44-mediated sequestration of HMW adiponectin in the ER, allowing secretion of HMW adiponectin from cells.

Without wishing to be bound by any theory, the present inventors believe that peptides of the invention bind ERp44 via Cys29. Various predictive tools well known to those skilled in the art may be used to identify putative ERp44 binding domains or motifs in ERp44 client proteins predicted to bind to Cys29. In one embodiment the cargo peptide may be derived from an ERp44 binding domain, including any putative ERp44 binding domain, derived from any protein.

In various embodiments cargo peptides for use in the invention may comprise 1, 2, 3, 4, 5, 6, 7, 8, 9 or 10 or more cysteine residues, and ranges may be selected between any of these values, for example, from about 1 to about 10, 1 to about 7, 1 to about 5, 1 to about 4, 1 to about 3 or from about 1 to about 2 cysteine residues.

In various embodiments the cargo peptide may be derived from the putative ERp44 binding domains of PrX4, SERT, IL-2, the tail region of ERp44 or ERO1α. In various embodiments the cargo peptide may comprise three or more, four or more, five or more or six or more contiguous amino acids from the SERT C109 ERp44 binding domain, SERT C200 ERp44 binding domain, SERT-C209 ERp44 binding domain, PrX4-C208 ERp44 binding domain, IL-2 C78 ERp44 binding domain, IL-2 C125 ERp44 binding domain, IL-2 C145 ERp44 binding domain, ERp44 tail C369 binding domain or the ERO1α C94-C99-C104 ERp44 binding domain. In one embodiment the cargo peptide may be derived from the adiponectin ERp44 binding domain.

In various embodiments one or more amino acids in the ERp44 binding domain amino acid sequence may be substituted for a naturally occurring amino acid or a non-naturally occurring amino acid. In various embodiments the amino acid substitution may be a conservative substitution or a non-conservative substitution.

In various embodiments the cargo peptide may comprise two or more, three or more, four or more or five or more cargo peptides. In various embodiments the peptides may comprise two or more repeated cargo peptides or two or more distinct cargo peptides. In various embodiments the two or more cargo peptides may be linked by a linker described herein.

### Cell Penetrating Agents

The peptides of the invention comprise one or more cell penetrating agents. As used herein, the term “cell penetrating agent” refers to an agent that facilitates transport of the cargo peptide across a cell membrane.

In one embodiment the cell penetrating agent comprises a cell penetrating peptide (CPP).

In various embodiments the cell penetrating peptide may comprise a cationic cell penetrating peptide, an amphipathic cell penetrating peptide or a hydrophobic cell penetrating peptide. A list of non-limiting examples of cell penetrating peptides that can be used are listed in Table 1 below. Other examples are described in Guidotti et al., 2017. Trends in Pharmacol. Sci. 38: 406-424.

TABLE 1
Cell penetrating peptides and variants

| CPP | Amino acid sequence | SEQ ID NO: |
|---|---|---|
| TAT | YGRKKRRQRRR | 26 |
| | YGRKKRRQQRRR | 27 |
| | YGRKKRR | 28 |
| | GRKKRRQRRRPPQ | 29 |
| | RKKRRQRRR | |
| Penetratin (Antp) | RQIKIWFQNRRMKWKK | 30 |
| DPV1047 | VKRGLKLRHVRPRVTRMDV | 31 |
| Bac | RRIRPRPPRLPRPRPRPLPFPRPG | 32 |
| SynB1 | RGGRLSYSRRRFSTSTGR | 33 |
| SynB1-NLS | RGGRLSYSRRRFSTSTGRWSQPKKKRKV | 34 |
| Poly-arginine | (R)₇₋₁₁ | |
| VP22 | DAATATRGRSAASRPTQRPRAPARSASRPRRPVQ | 35 |
| | NAKTRRHERRRKLAIER | 36 |
| Transportan | GWTLNSAGYLLGKINLKALAALAKKIL | 37 |
| MAP | KLALKLALKALKAALKLA | 38 |
| pVEC | LLIILRRRIRKQAHAHSK | 39 |
| MTS | AAVALLPAVLLALLAP | 40 |
| hCT derived | LGTYTQDFNKFHTFPQTAIGVGAP | 41 |
| MPG | GALFLGFLGAAGSTMGAWSQPKKKRKV | 42 |
| | GALFLGFLGAAGSTMGA | 43 |
| Buforin 2 | TRSSRAGLQFPVGRVHRLLRK | 44 |
| PEP-1 | KETWWETWWTEWSQPKKKRKV | 45 |
| Magainin 2 | GIGKFLHSAKKFGKAFVGEIMNS | 46 |
| M918 | MVTVLFRRLRIRRACGPPRVRV | 47 |
| C105Y | CSIPPEVKFNKPFVYLI | 48 |
| PFVYLI | PFVYLI | 49 |
| pVEC | LLIILRRRIRKQAHAHSK | 50 |
| BPrPR (1-28) | MVKSKIGSWILVLFVAMWSDVGLCKKRP | 51 |
| ARF (1-22) | MVRRFLVTLRIRRACGPPRVRV | 52 |
| p28 | LSTAADMQGVVTDGMASGLDKDYLKPDD | 53 |
| VT5 | DPKGDPKGVTVTVTVTVTGKGDPKPD | 54 |
| YTA2 | YTAIAWVKAFIRKLRK | 55 |
| YTA4 | IAWVKAFIRKLRKGPLG | 56 |
| CADY | GLWRALWRLLRSLWRLLWRA | 57 |
| Pep-7 | SDLWEMMMVSLACQY | 58 |

In various embodiments one or more amino acids in the cell penetrating peptide amino acid sequence may be substituted for a naturally occurring amino acid or a non-naturally occurring amino acid. In various embodiments the amino acid substitution may be a conservative substitution or a non-conservative substitution.

In various embodiments the cell penetrating peptide may be modified. Examples of suitable modifications include replacement of lysine residues with ornithine residues or modification of the side chain of one or more amino acids in the cell penetrating peptide. Such modifications may provide advantages including improved resistance of the peptide to protease degradation or faster cell penetration.

In embodiment the cell penetrating peptide may be a cyclic cell penetrating peptide. In various embodiments the cell penetrating peptide may be a cyclic TAT cell penetrating peptide or a cyclic polyarginine cell penetrating peptide.

Methods to identify suitable cell penetrating agents, including methods of measuring cell membrane permeation and methods to improve membrane permeability are well known in the art, such as those described in Yang and Hinner, 2015. Methods Mol. Biol. 1266: 29-53.

In one embodiment the cell penetrating agent may be a cell penetrating peptoid. In one embodiment the cell penetrating peptoid may be an oligomer of N-substituted glycine units.

### Linker

The peptides of the invention comprise a linker binding the cell penetrating agent and the cargo peptide. Further linkers may be present between first and second and between second and subsequent cargo peptides in peptides comprising two or more cargo peptides.

In various embodiments the linker may comprise a simple covalent bond, a flexible peptide linker, or a chemical spacer. In one embodiment the linker may be a polymer such as polyethylene glycol (PEG). In one embodiment the linker may be a peptide bond. Peptide linkers may be entirely artificial (e.g., comprising 2 to 20 amino acid residues independently selected from the group consisting of glycine, serine, asparagine, threonine, leucine and alanine) or adopted from naturally occurring proteins. Disulfide bridge formation can be achieved, e.g., by addition of cysteine residues, as further described herein below.

Selection of linkers should take into account that the linker should not substantially interfere with the ability of the cargo peptide to enhance adiponectin release or the ability of the cell penetrating agent to traverse the cell membrane. Linkers may be selected to improve folding and/or stability of the peptide and/or to target the peptide to the endoplasmic reticulum.

In one embodiment the linker may be a moiety which is covalently attached to a side chain, the N-terminus or the C-terminus of the cargo peptide. In one embodiment the linker may be a moiety which is covalently attached to a side chain, the N-terminus or the C-terminus of the cell penetrating peptide.

In one embodiment the linker may be attached to the C-terminus of the cargo peptide and the N-terminus of the cell penetrating peptide. In another embodiment the linker may be attached to the N-terminus of the cargo peptide and to the C-terminus of the cell penetrating peptide.

In one embodiment the linker may comprise additional amino acids from the cargo peptide sequence. For example, in one embodiment where the cargo peptide comprises an amino acid sequence derived from an ERp44 binding domain, the linker may comprise additional amino acids from the ERp44 binding domain sequence.

In one embodiment the linker may comprise additional amino acids linked together by peptide bonds which serve as spacers such that the linker does not interfere with the biological activity of the peptide. The linker is preferably made up of amino acids linked together by peptide bonds. Thus, in preferred embodiments, the linker may comprise from 1 to 10 amino acids linked by peptide bonds, wherein the amino acids are selected from the 20 naturally occurring amino acids.

In one embodiment the linker may comprise one or more amino acids selected from the group comprising glycine, alanine, proline, asparagine and lysine. Preferably, the linker is made up of a majority of amino acids that are sterically unhindered, such as glycine and alanine.

Other suitable linkers are well known and will be apparent to those skilled in the art. Examples of other linkers that may be suitable for use in the peptides are described in Hu et al., 2016. Chem. Soc. Rev. 45, 1691.

### Peptide Properties

ERp44 exploits the endoplasmic reticulum (ER)-Golgi pH gradient to bind adiponectin in the acidic cis-Golgi and release it in the neutral ER environment.

In a preferred embodiment, a peptide of the invention may bind ERp44 at a pH of about 6.5 and release ERp44 at a pH of about 8. The ERp44 binding profile of a given peptide can be assessed using a peptide release assay as described in the Examples below.

In one embodiment the amount of peptide-ERp44 complex present at pH 8.0 may be less than about 90%, 80%, 70%, 60%, 50%, 40%, 30%, 20% or less than about 10% of the amount of peptide-ERp44 complex present at pH 6.5 as determined by incubating the peptide with ERp44 at a ratio of 10:1 at pH 6.5 and increasing the pH to 8.0 and comparing the degree of ERp44-peptide complexation at pH 6.5 and pH 8.0.

The ability of peptides of the invention to increase adiponectin secretion from adipocytes or adipose tissue can be assessed using experimental methods described herein.

In one exemplary method, adipocytes (for example 3T3-L1 adipocytes) are incubated with a peptide comprising the cargo peptide at a concentration of 200 nM for 24 hours. Total adiponectin in the culture medium of the cells is determined and compared to total adiponectin in the culture medium of 3T3-L1 cells incubated without the peptide or with a control peptide such as the cargo peptide alone under the same conditions. Adiponectin may be determined and measured using methods known in the art and described herein, for example, immunoprecipitation and Western blotting using an anti-adiponectin antibody.

In various embodiments the peptide may induce an increase in total secreted adiponectin of at least about 10%, 15%, 20%, 25%, 30%, 35%, 40%, 45%, 50%, 55%, 60%, 65%, 70%, 75%, 80%, 85%, 90%, 95% or at least about 100%.

In various embodiments the peptide may induce an increase in HMW secreted adiponectin of at least about 10%, 15%, 20%, 25%, 30%, 35%, 40%, 45%, 50%, 55%, 60%, 65%, 70%, 75%, 80%, 85%, 90%, 95% or at least about 100%.

The suitability of peptides for use in the invention can be assessed in vivo, for example, by administering peptides to mice and assessing adiponectin levels in tissue or serum. In one embodiment, a peptide comprising the cargo peptide is administered to mice and serum samples at a suitable interval after administration, for example, four weeks. Serum adiponectin concentrations may be measured using ELISA with an anti-adiponectin antibody as described below.

In various embodiments the peptides of the invention may induce a transient inactivation of ERp44 or a transient inhibition of ERp44 binding to adiponectin.

### Peptide Synthesis

In one embodiment the peptide may comprise a synthetic peptide. Synthetic peptides may be prepared using solid phase peptide synthesis (SPPS).

The basic principle for solid phase peptide synthesis (SPPS) is a stepwise addition of amino acids to a growing polypeptide chain anchored via a linker molecule to a solid phase support, typically a resin particle, which allows for cleavage and purification once the polypeptide chain is complete. Briefly, a solid phase resin support and a starting amino acid are attached to one another via a linker molecule. Such resin-linker-acid matrices are commercially available.

The amino acid to be coupled to the resin is protected at its Nα-terminus by a chemical protecting group.

The amino acid may also have a side-chain protecting group. Such protecting groups prevent undesired or deleterious reactions from taking place during the process of forming the new peptide bond between the carboxyl group of the amino acid to be coupled and the unprotected Nα-amino group of the peptide chain attached to the resin.

The amino acid to be coupled is reacted with the unprotected Nα-amino group of the N-terminal amino acid of the peptide chain, increasing the chain length of the peptide chain by one amino acid. The carboxyl group of the amino acid to be coupled may be activated with a suitable chemical activating agent to promote reaction with the Nα-amino group of the peptide chain. The Nα-protecting group of N-terminal amino acid of the peptide chain is then removed in preparation for coupling with the next amino acid residue. This technique consists of many repetitive steps making automation attractive whenever possible. Those skilled in the art will appreciate that peptides may be coupled to the Nα-amino group of the solid phase bound amino acid or peptide instead of an individual amino acid, for example where a convergent peptide synthesis is desired.

When the desired sequence of amino acids is achieved, the peptide is cleaved from the solid phase support at the linker molecule.

SPPS may be carried out using a continuous flow method or a batch flow method. Continuous flow permits real-time monitoring of reaction progress via a spectrophotometer, but has two distinct disadvantages—the reagents in contact with the peptide on the resin are diluted, and scale is more limited due to physical size constraints of the solid phase resin. Batch flow occurs in a filter reaction vessel and is useful because reactants are accessible and can be added manually or automatically.

Two types of protecting groups are commonly used for protecting the N-alpha-amino terminus: “Boc” (tert-butyloxycarbonyl) and “Fmoc” (9-fluorenylmethyloxycarbonyl). Reagents for the Boc method are relatively inexpensive, but they are highly corrosive and require expensive equipment and more rigorous precautions to be taken. The Fmoc method, which uses less corrosive, although more expensive, reagents is typically preferred.

For SPPS, a wide variety of solid support phases are available. The solid phase support used for synthesis can be a synthetic resin, a synthetic polymer film or a silicon or silicate surface (e.g. controlled pore glass) suitable for synthesis purposes. Generally, a resin is used, commonly polystyrene suspensions, or polystyrene-polyethyleneglycol, or polymer supports for example polyamide. Examples of resins functionalized with linkers suitable for Boc-chemistry include PAM resin, oxime resin SS, phenol resin, brominated Wang resin and brominated PPOA resin. Examples of resins suitable for Fmoc chemistry include amino-methyl polystyrene resins, AMPB-BHA resin, Sieber amide resin, Rink acid resin, Tentagel S AC resin, 2-chlorotrityl chloride resin, 2-chlorotrityl alcohol resin, TentaGel S Trt-OH resin, Knorr-2-chlorotrityl resin, hydrazine-2-chlorotrityl resin, ANP resin, Fmoc photolable resin, HMBA-MBHA resin, TentaGel S HMB resin, Aromatic Safety Catch resinBAl resin and Fmoc-hydroxylamine 2 chlorotrityl resin. Other resins include PL CI-Trt resin, PL-Oxime resin and PL-HMBA Resin. Generally resins are interchangeable.

For each resin appropriate coupling conditions are known in the literature for the attachment of the starting monomer or sub-unit.

Preparation of the solid phase support includes solvating the support in an appropriate solvent (e.g. dimethylformamide). The solid phase typically increases in volume during solvation, which in turn increases the surface area available to carry out peptide synthesis.

A linker molecule is then attached to the support for connecting the peptide chain to the solid phase support. Linker molecules are generally designed such that eventual cleavage provides either a free acid or amide at the C-terminus. Linkers are generally not resin-specific. Examples of linkers include peptide acids for example 4-hydroxymethylphenoxyacetyl-4-methylbenzyhydrylamine (HMP), or peptide amides for example benzhydrylamine derivatives.

The first amino acid of the peptide sequence may be attached to the linker after the linker is attached to the solid phase support or attached to the solid phase support using a linker that includes the first amino acid of the peptide sequence. Linkers that include amino acids are commercially available.

The next step is to deprotect the Nα-amino group of the first amino acid. For Fmoc SPPS, deprotection of the Nα-amino group may be carried out with a mild base treatment (piperazine or piperidine, for example). Side-chain protecting groups may be removed by moderate acidolysis (trifluoroacetic acid (TFA), for example). For Boc SPPS, deprotection of the Nα-amino group may be carried out using for example TFA.

Following deprotection, the amino acid chain extension, or coupling, proceeds by the formation of peptide bonds. This process requires activation of the C-α-carboxyl group of the amino acid to be coupled. This may be accomplished using, for example, in situ reagents, preformed symmetrical anhydrides, active esters, acid halides, or urethane-protected N-carboxyanhydrides. The in situ method allows concurrent activation and coupling. Coupling reagents include carbodiimide derivatives, for example N,N′-dicyclohexylcarbodiimide or N,N-diisopropylcarbodiimide. Coupling reagents also include uronium or phosphonium salt derivatives of benzotriazol. Examples of such uronium and phosphonium salts include HBTU (O-1H-benzotriazole-1-yl)-N,N,N′,N′-tetramethyluronium hexafluorophosphate), BOP (benzotriazole-1-yl-oxy-tris-(dimethylamino)-phosphonium hexafluorophosphate), PyBOP (Benzotriazole-1-yl-oxy-tripyrrolidinophosphonium hexafluorophosphate), PyAOP, HCTU (O-(1H-6-chloro-benzotriazole-1-yl)-1,1,3,3-tetramethyluronium hexafluorophosphate), TCTU (O-1H-6-chlorobenzotriazole-1-yl)-1,1,3,3-tetramethyluronium tetrafluoroborate), HATU (O-(7-azabenzotriazol-1-yl)-1,1,3,3-tetramethyluronium hexafluorophosphate), TATU (O-(7-azabenzotriazol-1-yl)-1,1,3,3-tetramethyluronium tetrafluoroborate), TOTU (O-[cyano(ethoxycarbonyl)methyleneamino]-N,N,N′,N″-tetramethyluronium tetrafluoroborate), and HAPyU (O-(benzotriazol-1-yl)oxybis-(pyrrolidino)-uronium hexafluorophosphate. In some embodiments, the coupling reagent is HBTU, HATU, BOP, or PyBOP.

After the desired amino acid sequence has been synthesized, the peptide is cleaved from the resin. The conditions used in this process depend on the sensitivity of the amino acid composition of the peptide and the side-chain protecting groups. Generally, cleavage is carried out in an environment containing a plurality of scavenging agents to quench the reactive carbonium ions that originate from the protective groups and linkers. Common cleaving agents include, for example, TFA and hydrogen fluoride (HF). In some embodiments, where the peptide is bound to the solid phase support via a linker, the peptide chain is cleaved from the solid phase support by cleaving the peptide from the linker.

The conditions used for cleaving the peptide from the resin may concomitantly remove one or more side-chain protecting groups.

The use of protective groups in SPPS is well established. Examples of common protective groups include but are not limited to acetamidomethyl (Acm), acetyl (Ac), adamantyloxy (AdaO), benzoyl (Bz), benzyl (Bzl), 2-bromobenzyl, benzyloxy (BzlO), benzyloxycarbonyl (Z), benzyloxymethyl (Bom), 2-bromobenzyloxycarbonyl (2-Br-Z), tert-butoxy (tBuO), tert-butoxycarbonyl (Boc), tert-butoxymethyl (Bum), tert-butyl (tBu), tert-buthylthio (tButhio), 2-chlorobenzyloxycarbonyl (2-CI-Z), cyclohexyloxy (cHxO), 2,6-dichlorobenzyl (2,6-DiCl-Bzl), 4,4′-dimethoxybenzhydryl (Mbh), 1-(4,4-dimethyl-2,6-dioxo-cyclohexylidene)3-methyl-butyl (ivDde), 4-{N41-(4,4-dimethyl-2,6-dioxo-cyclohexylidene)3-methylbutylFamino) benzyloxy (ODmab), 2,4-dinitrophenyl (Dnp), fluorenylmethoxycarbonyl (Fmoc), formyl (For), mesitylene-2-sulfonyl (Mts), 4-methoxybenzyl (MeOBzl), 4-methoxy-2,3,6-trimethyl-benzenesulfonyl (Mtr), 4-methoxytrityl (Mmt), 4-methylbenzyl (MeBzI), 4-methyltrityl (Mtt), 3-nitro-2-pyridinesulfenyl (Npys), 2,2,4,6,7-pentamethyldihydrobenzofurane-5-sulfonyl (Pbf), 2,2,5,7,8-pentamethyl-chromane-6-sulfonyl (Pmc), tosyl (Tos), trifluoroacetyl (Tfa), trimethylacetamidomethyl (Tacm), trityl (Trt) and xanthyl (Xan).

Where one or more of the side chains of the amino acids of the peptide contains functional groups, such as for example additional carboxylic, amino, hydroxy or thiol groups, additional protective groups may be necessary. For example, if the Fmoc strategy is used, Mtr, Pmc, Pbf may be used for the protection of Arg; Trt, Tmob may be used for the protection of Asn and Gln; Boc may be used for the protection of Trp and Lys; tBu may be used for the protection of Asp, Glu, Ser, Thr and Tyr; and Acm, tBu, tButhio, Trt and Mmt may be used for the protection of Cys. A person skilled in the art will appreciate that there are numerous other suitable combinations.

The methods for SPPS outlined above are well known in the art. See, for example, Atherton and Sheppard, “Solid Phase Peptide Synthesis: A Practical Approach,” New York: IRL Press, 1989; Stewart and Young: “Solid-Phase Peptide Synthesis 2nd Ed.,” Rockford, Ill.: Pierce Chemical Co., 1984; Jones, “The Chemical Synthesis of Peptides,” Oxford: Clarendon Press, 1994; Merrifield, J. Am. Soc. 85:2146-2149 (1963); Marglin, A. and Merrifield, R. B. Annu. Rev. Biochem. 39:841-66 (1970); and Merrifield R. B. JAMA. 210(7):1247-54 (1969); and “Solid Phase Peptide Synthesis—A Practical Approach” (W. C. Chan and P. D. White, eds. Oxford University Press, 2000). Equipment for automated synthesis of peptides or polypeptides is readily commercially available from suppliers such as Perkin Elmer/Applied Biosystems (Foster City, Calif.) and may be operated according to the manufacturer's instructions.

Following cleavage from the resin, the peptide may be separated from the reaction medium, e.g. by centrifugation or filtration. The peptide may then be subsequently purified, e.g. by HPLC using one or more suitable solvents.

In another embodiment the peptide may be produced by liquid-phase synthesis.

In another embodiment the peptide may be produced recombinantly. Methods of producing recombinant peptides are well known to those skilled in the art.

### 3. Therapeutic Outcomes and Methods of Assessing Therapeutic Outcomes

Adiponectin plays a protective role in obesity-related diseases. In obese subjects, serum levels of adiponectin, particularly the active HMW form of adiponectin, are markedly reduced.

The present inventors have shown that administration of the peptides described herein increases serum levels of adiponectin, particularly HMW adiponectin, in a subject.

The present invention relates to a method of treating or preventing a condition associated with
adiponectin dysfunction,
obesity,
weight gain,
reduced serum levels of adiponectin,
reduced insulin sensitivity,
increased serum cholesterol,
increased serum triglycerides, and/or
increased blood glucose,
   in a subject in need thereof.

In various embodiments the peptides or compositions described herein may be useful for treating or preventing conditions selected from the group comprising metabolic syndrome, insulin resistance, diabetes, musculoskeletal disease, musculoskeletal disease, cardiovascular disease, respiratory disease, gallbladder disease, liver disease, gynaecological disease, sexual dysfunction, neurodegenerative disease or cancer. In various embodiments the peptides or compositions described herein may be useful for treating or preventing type 2 diabetes, hypertension, hypercholesterolemia, hypertriglyceridemia, osteoarthritis, atherosclerosis, heart attack, stroke, sleep apnoea, nonalcoholic fatty liver disease, infertility, irregular menstruation, erectile dysfunction, Alzheimer's disease, Parkinson's disease, endometrial cancer, uterine cancer, breast cancer, ovarian cancer, prostate cancer, liver cancer, gallbladder cancer, kidney cancer, rectal cancer, oesophageal cancer, gallbladder cancer and colon cancer.

In various embodiments the subject may be overweight or obese, or at risk of becoming overweight or obese.

In various embodiments the subject may have a BMI greater than 25, greater than 26, greater than 27, greater than 28, greater than 29 or a BMI greater than 30.

The efficacy of a peptide or composition can be evaluated both in vitro and in vivo. For example, the composition can be tested in vitro or in vivo for its ability to induce secretion of adiponectin from cells as described herein. For in vivo studies, the peptide or composition can be fed to or injected into an animal (e.g., a mouse) and its effects on, for example, blood glucose levels, blood cholesterol levels, blood triglyceride levels, insulin sensitivity and/or diabetes are then assessed. Based on the results, an appropriate dosage range and administration route can be determined.

### 4. Gene Therapy

The present invention also relates to a method of treating or preventing a disease associated with adiponectin dysfunction, the method comprising administering to a subject a gene therapy vector comprising a nucleotide sequence encoding a cargo peptide described herein wherein the vector induces expression of a therapeutically effective amount of the cargo peptide.

In one embodiment expression of the nucleotide sequence encoding the cargo peptide gene may be under the control of a regulatory sequence that induces tissue-specific or cell-specific expression of the cargo peptide. In an exemplary embodiment the regulatory sequence may induce expression of the cargo peptide in adipocytes.

An appropriate regulatory sequence, for example a tissue-specific promoter, can be readily selected by those skilled in the art for a given target tissue. For example, to induce expression of the cargo peptide in adipose tissue, an adipose-specific regulatory sequence can be used.

The gene therapy vector may or may not comprise an origin of replication.

In various embodiments the gene therapy vector may be administered in a form to enhance delivery to target cells or tissues. For example, in various embodiments the gene therapy vector may be delivered in the form of a lipoplex, polymersome, polyplex, dendrimer, or nanoparticle. In one embodiment the gene therapy vector may be linked to one or more cell penetrating peptides.

### 5. Pharmaceutical Compositions

The present invention also relates to a pharmaceutical composition comprising an effective amount of a peptide described herein or a pharmaceutically acceptable salt or solvent thereof, and a pharmaceutically acceptable carrier.

The pharmaceutical compositions may comprise an effective amount of two or more peptides described herein in combination.

Pharmaceutically acceptable carriers that may be used in the compositions include, but are not limited to, ion exchangers, alumina, aluminum stearate, lecithin, self-emulsifying drug delivery systems (SEDDS) such as d-α-tocopherol polyethyleneglycol 1000 succinate, surfactants used in pharmaceutical dosage forms such as Tweens or other similar polymeric delivery matrices, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Cyclodextrins such as α-, β-, and γ-cyclodextrin, or chemically modified derivatives such as hydroxyalkylcyclodextrins, including 2- and 3-hydroxypropyl-β-cyclodextrins, or other solubilized derivatives may also be advantageously used to enhance delivery. Oil solutions or suspensions may also contain a long-chain alcohol diluent or dispersant, or carboxymethyl cellulose or similar dispersing agents, which are commonly used in the formulation of pharmaceutically acceptable dosage forms such as emulsions and or suspensions.

The compositions are formulated to allow for administration to a subject by any chosen route, including but not limited to oral or parenteral (including topical, subcutaneous, intramuscular and intravenous) administration.

For example, the compositions may be formulated with an appropriate pharmaceutically acceptable carrier (including excipients, diluents, auxiliaries, and combinations thereof) selected with regard to the intended route of administration and standard pharmaceutical practice. For example, the compositions may be administered orally as a powder, liquid, tablet or capsule, or topically as an ointment, cream or lotion. Suitable formulations may contain additional agents as required, including emulsifying, antioxidant, flavouring or colouring agents, and may be adapted for immediate-, delayed-, modified-, sustained-, pulsed- or controlled-release.

The compositions may be formulated to optimize bioavailability, immunogenicity, or to maintain plasma, blood, or tissue concentrations within the immunogenic or therapeutic range, including for extended periods. Controlled delivery preparations may also be used to optimize the peptide concentration at the site of action, for example.

The compositions may be formulated for periodic administration, for example to provide continued exposure.

The compositions may be administered via the parenteral route. Examples of parenteral dosage forms include aqueous solutions, isotonic saline or 5% glucose of the active agent, or other well-known pharmaceutically acceptable excipients. Cyclodextrins, for example, or other solubilising agents well-known to those familiar with the art, can be utilized as pharmaceutical excipients for delivery of the therapeutic agent.

Examples of dosage forms suitable for oral administration include, but are not limited to tablets, capsules, lozenges, or like forms, or any liquid forms such as syrups, aqueous solutions, emulsions and the like, capable of providing a therapeutically effective amount of the composition. Capsules can contain any standard pharmaceutically acceptable materials such as gelatin or cellulose. Tablets can be formulated in accordance with conventional procedures by compressing mixtures of the active ingredients with a solid carrier and a lubricant. Examples of solid carriers include starch and sugar bentonite. Active ingredients can also be administered in a form of a hard shell tablet or a capsule containing a binder, e.g., lactose or mannitol, a conventional filler, and a tabletting agent. Dosage forms for oral administration can be formulated with an enteric coating to prevent dissolution or disintegration of the dosage form in the stomach to provide for delayed release of the peptide and/or to allow release of the peptide after the stomach (such as in the upper tract of the intestine).

Examples of dosage forms suitable for transdermal administration include, but are not limited, to transdermal patches, transdermal bandages, and the like.

Examples of dosage forms suitable for topical administration of the compositions include any lotion, stick, spray, ointment, paste, cream, gel, etc., whether applied directly to the skin or via an intermediary such as a pad, patch or the like.

Examples of dosage forms suitable for suppository administration of the compositions include any solid dosage form inserted into a bodily orifice particularly those inserted rectally, vaginally and urethrally.

Examples of dosage of forms suitable for injection of the compositions include delivery via bolus such as single or multiple administrations by intravenous injection, subcutaneous, subdermal, and intramuscular administration or oral administration.

Examples of dosage forms suitable for depot administration of the compositions and include pellets of the peptide or solid forms wherein the peptide is entrapped in a matrix of biodegradable polymers, microemulsions, liposomes or are microencapsulated.

Examples of infusion devices for the compositions include infusion pumps for providing a desired number of doses or steady state administration, and include implantable drug pumps.

Examples of implantable infusion devices for compositions include any solid form in which the peptide conjugates are encapsulated within or dispersed throughout a biodegradable polymer or synthetic, polymer such as silicone, silicone rubber, silastic or similar polymer.

Examples of dosage forms suitable for transmucosal delivery of the compositions include depositories solutions for enemas, pessaries, tampons, creams, gels, pastes, foams, nebulised solutions, powders and similar formulations containing in addition to the active ingredients such carriers as are known in the art to be appropriate. Such dosage forms include forms suitable for inhalation or insufflation of the compositions, including compositions comprising solutions and/or suspensions in pharmaceutically acceptable, aqueous, or organic solvents, or mixture thereof and/or powders. Transmucosal administration of the compositions may utilize any mucosal membrane but commonly utilizes the nasal, buccal, vaginal and rectal tissues. Formulations suitable for nasal administration of the compositions may be administered in a liquid form, for example, nasal spray, nasal drops, or by aerosol administration by nebulizer, including aqueous or oily solutions of the polymer particles. Formulations may be prepared as aqueous solutions for example in saline, solutions employing benzyl alcohol or other suitable preservatives, absorption promoters to enhance bio-availability, fluorocarbons, and/or other solubilising or dispersing agents known in the art.

Examples of dosage forms suitable for buccal or sublingual administration of the compositions include lozenges, tablets and the like. Examples of dosage forms suitable for opthalmic administration of the compositions include inserts and/or compositions comprising solutions and/or suspensions in pharmaceutically acceptable, aqueous, or organic solvents.

Examples of formulations of compositions may be found in, for example, Sweetman, S. C. (Ed.). Martindale. The Complete Drug Reference, 33rd Edition, Pharmaceutical Press, Chicago, 2002, 2483 pp.; Aulton, M. E. (Ed.) Pharmaceutics. The Science of Dosage Form Design. Churchill Livingstone, Edinburgh, 2000, 734 pp.; and, Ansel, H. C., Allen, L. V. and Popovich, N. G. Pharmaceutical Dosage Forms and Drug Delivery Systems, 7th Ed., Lippincott 1999, 676 pp. Excipients employed in the manufacture of drug delivery systems are described in various publications known to those skilled in the art including, for example, Kibbe, E. H. Handbook of Pharmaceutical Excipients, 3rd Ed., American Pharmaceutical Association, Washington, 2000, 665 pp. The USP also provides examples of modified-release oral dosage forms, including those formulated as tablets or capsules. See, for example, The United States Pharmacopeia 23/National Formulary 18, The United States Pharmacopeial Convention, Inc., Rockville Md., 1995 (hereinafter “the USP”), which also describes specific tests to determine the drug release capabilities of extended-release and delayed-release tablets and capsules. The USP test for drug release for extended-release and delayed-release articles is based on drug dissolution from the dosage unit against elapsed test time. Descriptions of various test apparatus and procedures may be found in the USP. Further guidance concerning the analysis of extended release dosage forms has been provided by the F.D.A. (See Guidance for Industry. Extended release oral dosage forms: development, evaluation, and application of in vitro/in vivo correlations. Rockville, Md.: Center for Drug Evaluation and Research, Food and Drug Administration, 1997).

Where two or more peptides are administered or used, the two or more peptides may be administered or used simultaneously, sequentially, or separately.

The invention consists in the foregoing and also envisages constructions of which the following gives examples only and in no way limit the scope thereof.

### EXAMPLE 1

This example describes the in vitro binding of peptides of the invention to ERp44.

### 1. Materials and Methods

### Materials

For peptide synthesis all solvents and reagents were used as supplied. O-(Benzotriazol-1-yl)-N,N,N′,N′-tetramethyluroniumhexafluorophosphate (HBTU), (O-(7-azabenzotriazol-1-yl)-N,N,N′,N′-tetramethyluroniumhexafluorophosphate) (HATU), and Stritylmercaptopropionic acid were purchased from GL Biochem (Shanghai, China). Dimethylformamide (DMF) (AR grade) and acetonitrile (HPLC grade) were purchased from Scharlau (Barcelona, Spain). N,N′-diisopropylethylamine (DIPEA), piperidine, piperazine, ethanedithiol (EDT), diisopropylcarbodiimide (DIC), triisopropylsilane (TIS), dimethylsulfide (DMS), tert-butylammonium iodide (TBAI) and phenylacetamidomethyl (PAM) linker were purchased from Aldrich (St Louis, Mo.) and N-methylpyrrolidine (NMP) was purchased from Fluka (Buchs, Switzerland). Trifluoroacetic acid (TFA) was purchased from Halocarbon (River Edge, N.J.). 1- Hydroxybenzotriazole hydrate (HOBt.H2O) was purchased from Advanced Chemtech (Louisville, Ky.). Anhydrous hydrogen fluoride was obtained from Matheson Trigas (Basking Ridge, N.J.). Aminomethyl polystyrene (AM-PS) resin was synthesized “in house” as described (Harris, Yang et al., 2011). Aminomethyl ChemMatrix resin was obtained from PCAS Biomatrix (Quebec, Canada). Fmoc-amino acids were purchased from GL Biochem. The cDNA encoding mouse ERp44 was purchased from OriGene Technologies (Rockville, USA). Restriction enzymes were from Roche Diagnostics N.Z., Ltd (New Zealand). DNA Oligonuclotides were from Integrated DNA Technologies Pte. Ltd. (Singapore). Other chemicals were from Sigma-Aldrich (St Louis, Mo.). All reagents for cell culture were purchased from Invitrogen Corporation (Grand Island, N.Y., USA). Consumables for Western blotting, including PVDF membrane, enhanced chemiluminescence (ECL) plus detection kit and ECL advance detection kit were purchased from GE Healthcare (Piscataway, N.J., USA). Medical X ray film was purchased from Fuji (Tokyo, Japan).

### Production of Peptides

Short cargo peptides and CPP-cargo peptides based on ERp44 binding domains from adiponectin and IgM (see Table 2) were synthesized manually using Fmoc/tBu solid phase synthesis in a fritted glass reaction vessel (see Amblard et al., 2006 Mol Biotechnol 33: 239-254.) on amino methyl polystyrene resin (Harris et al., 2011 Tetrahedron Letters 52: 6024-6026) equipped with the acid labile HMPP linker (Albericio & Barany, 1985 International Journal of Peptide and Protein Research 26: 92-97).

The N-Fmoc group was deprotected with 20% v/v piperidine in dimethyl formamide (DMF) twice for 10 minutes and coupling of individual amino acids was performed with 5.5 equivalents of Fmoc protected amino acid in DMF (0.2 M), 5 equivalents of O-(benzotriazol-1-yl)-N,N,N′,N″-tetramethyluroniumhexafluorophosphate (HBTU) in DMF (0.45 M) and 10 equivalents of diisopropylethylamine (DIPEA) in N-methylpyrrolidine (NMP) (2 M) for 30 min.

Upon completion of the synthesis the peptide was released from the resin with concomitant removal of protecting groups by treatment with trifluoroacetic acid/triisopropylsilane/H2 O/ethane dithiol (94:1:2.5:2.5, v/v/v/v) at room temperature for 3 h. The crude peptide was precipitated with ice cold diethyl ether, isolated by centrifugation, washed with cold diethyl ether, dissolved in 1:1 (v/v) acetonitrile:water containing 0.1% trifluoroacetic acid and lyophilized.

Purification using a solvent system of A (0.1% TFA in H2O) and B (0.1% TFA in acetonitrile) was performed by semi-prep RP HPLC (Dionex Ultimate 3000 equipped with a 4 channel UV detector) at 210 nm using a Gemini C18 (5 μm; 10×250 mm) column (Phenomenex) at 5.0 ml/min flow rate and eluting with an appropriate shallow gradient of increasing concentration of B.

Fractions were analyzed for purity by HPLC and/or MS, pooled, lyophilized and stored at −20° C. Purified peptides were analyzed for purity by analytical HPLC (Dionex Ultimate 3000 equipped with 4 channel UV detector) at 214 nm using a Zorbax Eclipse XDB-C8 (5 μm; 4.6×150 mm) column (Agilent) at 1 ml/min flow rate using a linear gradient of 5-65% over 21 mins at ca.3% B per minute. To remove trifluoroacetic acid (TFA) from the lyophilized peptide, the peptide was dissolved in 10 mM hydrochloride acid (10 mM) at a concentration of 1 mg/ml and again lyophilized. This procedure was repeated three times.

LC-MS was performed using an Agilent (Santa Clara, Calif.) 1100 Compact HPLC equipped with a single wavelength UV detector at 214 nm with an in-line Hewlett Packard (Palo Alto, Calif.) 1100MSD mass spectrometer using ESI in the positive mode. The solvent system used was A (0.1% formic acid in H2O) and B (0.1% formic acid in acetonitrile). An Agilent Zorbax 300SB-C3 3.5μ; 3.0×150 mm) column was used with a linear gradient of 5% to 65% B over 21 mins at 0.3 ml/min.

TABLE 2
Sequence, molecular weight, yield and purity of peptides.

| | | Molecular | | Seq |
|---|---|---|---|---|
| | | Weight | | ID |
| Peptide | Sequence | (Da) | Yield and purity | No. |
| Adiponectin | KGTCAGWMA | 924 | 22.1 mg, yield | 59 |
| | | | 24.0%, purity > 95% | |
| CPP- | YGRKKRRQQRRRKGTCAGWMA | 2466 | 45.3 mg, yield | 60 |
| adiponectin | | | 18.3%, purity > 95% | |
| M1 | GTCY | 441 | 14.3 mg, yield | 25 |
| | | | 32.4%, purity > 95% | |
| M2 | DTGGTCY | 715 | 21.8 mg, 30.4% | 24 |
| | | | yield%, purity > 95% | |
| CPP-M1 | YGRKKKRRQQRRRGTCY | 1984 | 32.1 mg, yield | 61 |
| | | | 16.2%, purity > 95% | |
| CPP-M2 | YGRKKKRRQQRRRDTGGTCY | 2557 | 35.8 mg, yield | 62 |
| | | | 14.0%, purity > 95% | |

### Production of ERp44

The cDNA encoding murine ERp44 without the signal sequence was amplified by PCR and cloned into the pET28b vector at the Nhel and Xhol sites. Recombinant ERp44 was overexpressed in E. coli and purified as described (Hampe et al., 2015 Journal of Biological Chemistry 290: 18111-18123). To separate ERp44 monomers and dimers, purified protein was loaded onto a Superdex-200 10/300 GL column (GE Healthcare, pre-equilibrated with 20 mM MES, 150 mM NaCl, pH 6.5). The fractions containing monomeric ERp44 as the main component were pooled, confirmed by non-reducing SDS-PAGE, and used for subsequent experiments.

### Electrospray Ionization Mass Spectrometry

Mass spectrometry was used to quantify the amount of disulfide linkage between ERp44 and the various peptides. For this purpose, ERp44 (43 μM) was incubated with 10-fold excess (430 μM) of individual peptide. All samples were incubated for 4 days in 20 mM MES, 150 mM NaCl at pH 6.5 and subsequently subjected to mass spectrometric analyses.

For LC-MS analysis, samples were diluted in 0.1% formic acid, and 10 μl was injected onto a 0.32×100-mm 3-μm Discovery Bio Wide pore C5 column (Supelco, Bellefonte, Pa.) and separated using the following gradient at a flow rate of 6 ml/min: 0-4 min 10% B; 24 min 70% B, 27 min 97% B, 30 min 97% B, 32 min 10% B, and 35 min 10% B where A was 0.1% formic acid in water and B was 0.1% formic acid in acetonitrile. The column eluate was ionized in the electrospray source of a QSTAR-XL quadrupole time-of-flight mass spectrometer (Applied Biosystems, Foster City, Calif.). A TOF-MS scan from 400 to 1600 m/z was performed. The resulting data were de-convoluted into protein molecular masses using the Bayesian Protein Reconstruct Tool within Analyst QS 1.1 (Applied Biosystems).

### 2. Results

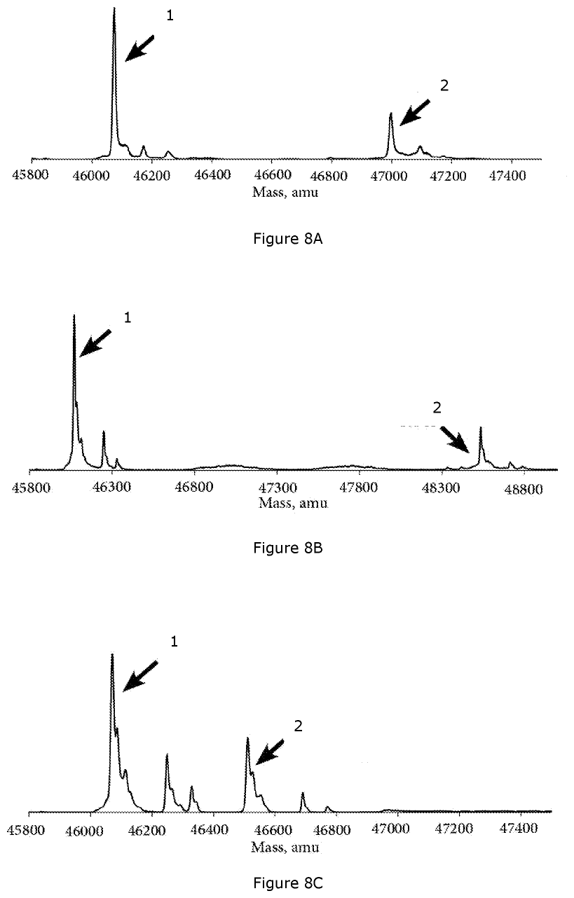

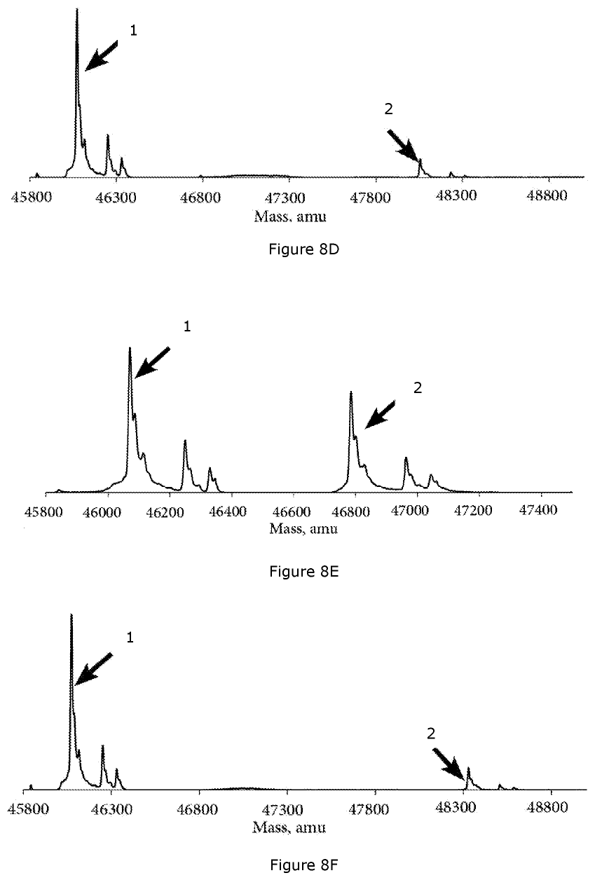

The results showed that all of the peptides tested formed a complex with ERp44 (FIG. 8).

### EXAMPLE 2

This example describes the effect of peptides of the invention on ERp44-adiponectin interactions and adiponectin production.

### 1. Methods

### Cell Culture

3T3-L1 cells (ATCC #CL-173, Manassas, Va., USA) were cultured in Dulbecco's Modified Eagle Medium (DMEM) supplemented with 10% fetal bovine serum (FBS) and 1% penicillin-streptomycin-fungisone (PSF) at 37° C. and under 5% CO₂ 95% humidified air. For differentiation, the confluent 3T3-L1 cultures were supplemented with 10 μg/ml insulin, 0.25 μM dexamethasone and 0.5 mM isobutylmethylxanthine for two days, followed by incubation with 10 μg/ml insulin in the culture medium for another two days. Subsequently, cells were incubated in DMEM with 10% FBS that was changed every two days. Peptide treatment (200 nM in FBS-free medium) was performed in fully differentiated 3T3-L1 adipocyte cultures. After incubation for 24 h, cell lysates and the conditioned medium were collected for subsequent analyses.

### Immunoprecipitation and Western Blotting

Cells or adipose tissues were washed with phosphate buffer (137 mM NaCl, 2.7 mM KCl, 4.3 mM Na2HPO4, 1.47 mM KH2PO4, pH 7.4) and then solubilized in lysis buffer (25 mM Tris-HCl pH 7.5, 150 mM NaCl, 5 mM NaF, 1% sodium deoxycholate, 1% Nonidet P-40, 1% Triton X-100 plus protease inhibitor mixtures). Equal amounts of lysates (100 μg) were precleared with protein G (Thermo Fisher Scientific, Waltham, Mass., USA) and incubated overnight with specific antibodies at 4° C. with shaking.

After incubating with protein G for another four hours, the immune-complexes were precipitated by low-speed centrifugation, washed three times with lysis buffer, and eluted in SDS-PAGE sample buffer (50 mM Tris-HCl pH 6.8, 1% SDS, 5% glycerol, 3% β-mercaptomethanol, 0.01% bromophenal blue) or 0.1 M glycine HCl (pH 3.0) and then diluted with non-reducing sample buffer (1% SDS, 5% glycerol, 10 mM Tris-HCl pH6.8) for analyzing adiponectin monomers and oligomers by Western blotting.

Cell lysates, media or serum samples were incubated with non-reducing sample buffer at room temperature for 10 minutes and then separated by 4-20% gradient SDS-PAGE. After transferring to polyvinylidene difluoride (PVDF) membranes, immunoblotting was performed using an in-house antibody against murine adiponectin developed by Antibody and Immunoassay Services, HKU (http://www.pharma.hku.hk/sweb/antibody/PolyclonalAntibodyCont.php?polyclonal_no=60 0005), or antibodies against ERp44 (Santa Cruz Biotechnology, Santa Cruz, Calif.), DsbA-L (Abcam, Cambridge, Mass., USA). Protein bands were quantified by ImageJ and intensities were compared. All experiments were repeated at least six times.

### 2. Results

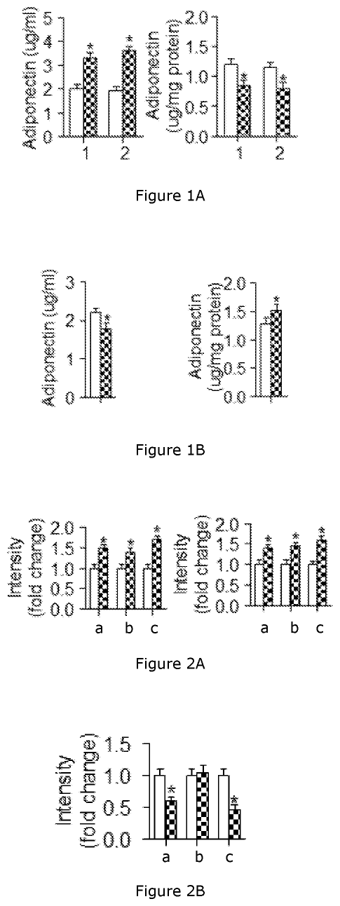

Treatment with CPP-M1 or CPP-M2 significantly increased the total amount of adiponectin secreted in the media (by ˜45% and ˜53% respectively), but decreased intracellular adiponectin levels (FIG. 1A). The total amount of adiponectin in the conditioned media was significantly reduced after incubation with CPP-adiponectin (by ˜16%) (FIG. 1B), but intracellular adiponectin levels were increased.

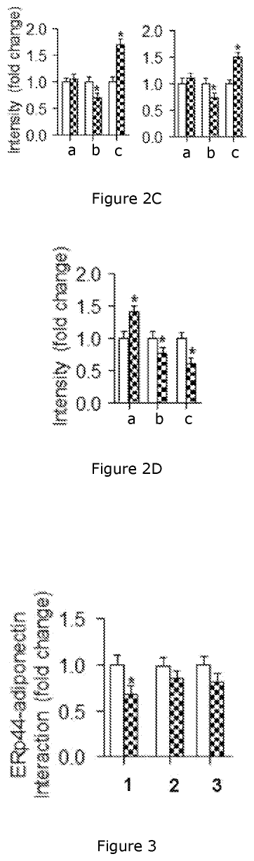

Non-reducing SDS-PAGE was performed to analyze the oligomeric distribution of adiponectin in the conditioned media. The results are shown in FIG. 2. Levels of all three forms of adiponectin (trimer, hexamer and HMW) were significantly elevated after treatment with CPP-M1 or CPP-M2 (FIG. 2A). The amount of trimeric and HMW adiponectin was significantly reduced in the media collected from CPP-adiponectin-treated cells (FIG. 2B).

CPP-M1 and CPP-M2 significantly increased the amount of HMW adiponectin, but decreased a sub-population of the hexametric adiponectin in the cell lysate when compared to those treated with IgM-derived cargo peptides (FIG. 2C). In cells treated with CPP-adiponectin, the oligomeric composition was shifted towards the trimer (FIG. 2D).

Co-immunoprecipitation was performed to determine the amount of adiponectin associated with ERp44. The results are shown in FIG. 3.

### EXAMPLE 3

This example describes the effect of peptides of the invention on HMW adiponectin levels in obese mice.

### 1. Methods

### Animal Studies

All experimental protocols were approved by the Committee on the Use of Live Animals for Teaching and Research of the University of Hong Kong and carried out in accordance with ARRIVE (Animal Research: Reporting of In Vivo Experiments) as well as institutional guidelines for the care and use of laboratory animals. Mice of the C57BL/6J background were housed in a room under controlled temperature (23±1° C.) with 12 hour light-dark cycles and free access to water and food. Mice were fed with either standard chow (STC; 3.07 kcal/g, LabDiet 5053, Purina Mills, Richmond, Ind., USA) or high-fat diet (HFD; 4.73 kcal/g containing 45% fat, 20% protein and 35% carbohydrate, D12451, Research diet, N.J., USA) starting from four-weeks old.

Immunoprecipitation and Western blotting studies were performed on serum adipose tissue samples obtained from mice as described above in Example 2. The serum was collected 24 hours after injection.

### 2. Results

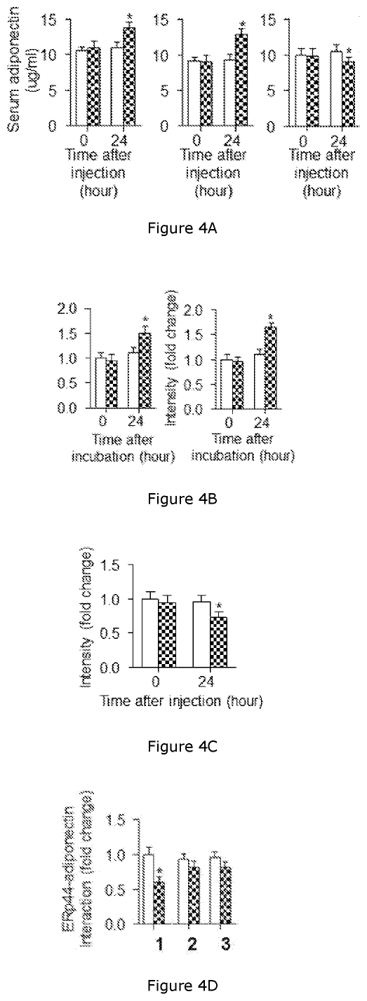

The circulating concentration of adiponectin was significantly increased in mice treated with CPP-M1 (by ˜28%) or CPP-M2 (by ˜34%), but decreased in those treated with CPP-adiponectin (by ˜14%), when compared to the corresponding cargo peptide only treatment groups (FIG. 4A). The HMW form of adiponectin was increased in mice treated with CPP-M1 or CPP-M2 (FIG. 4B). HMW adiponectin was significantly decreased in serum samples of mice treated with CPP-adiponectin (FIG. 4C).

The interactions between adiponectin and ERp44 in mouse adipose tissue were subsequently analyzed. CPP-adiponectin significantly decreased the amount of adiponectin bound to ERp44 (FIG. 4D).

### EXAMPLE 4

This example describes the effect of peptides of the invention on insulin sensitivity and energy homeostasis in obese mice.

### 1. Methods

### Treatment of Obese Mice

Mice were administered intraperitoneally with the peptides described in Table 2 (10 mg/kg body weight for acute studies at 16-weeks old or 5 mg/kg/day for four-week chronic treatment at 12-weeks old). Body weights of the animals were monitored on a weekly basis. Fasting blood glucose was determined after 16-hours food starvation (from 6:00 pm to 10:00 am) by a Accu-Check Advantage II Glucometer (Roche Diagnostics Mannheim, Germany). Fasting body fat mass was determined by the minispec Body Composition Analyzer (Bruker Optics Inc., Tex., USA). Circulating and tissue contents of lipids, including triglycerides and total cholesterols, were analyzed using LiquiColor® Triglycerides and Stanbio Cholesterol assay kits, respectively. Serum adiponectin concentrations were determined with an in-house ELISA kit as described (Wang et al., 2006 J Biol Chem 281: 16391-16400). Serum insulin levels were measured with an in-house mouse insulin ELISA kit (Cat No. 32100, http://www.pharma.hku.hk/sweb/antibody/ELISA.php). Homeostatic Model Assessment of

Insulin Resistance (HOMA-IR) was calculated as fasting insulin (μU/ml)×fasting glucose (mmol/L)/22.5 for comparison as described (Xu et al., 2013). All results were compared between mice treated with control and CPP peptides.

### 2. Results

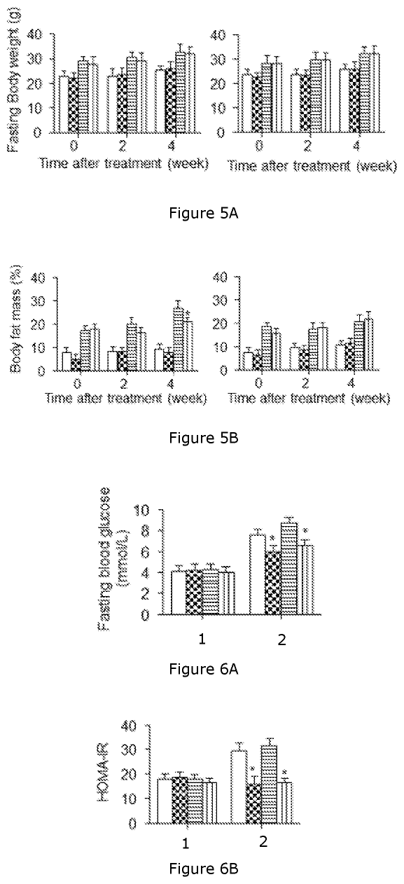

The results are shown in FIGS. 5-7. Fasting body weight was unchanged in all treatment groups over four weeks (FIG. 5A).

After four-weeks treatment, mice administered CPP-M1 exhibited a significantly decreased body fat mass compared to those administered M1 cargo peptide under HFD (FIG. 5B). Body fat mass was not significantly different in mice under STC, in treatment groups. Both CPP-M1 and CPP-M2 significantly blocked HFD-induced hyperglycemia and insulin resistance, as demonstrated by the fasting glucose levels and the calculated HOMA-IR (FIGS. 6A and 6B).

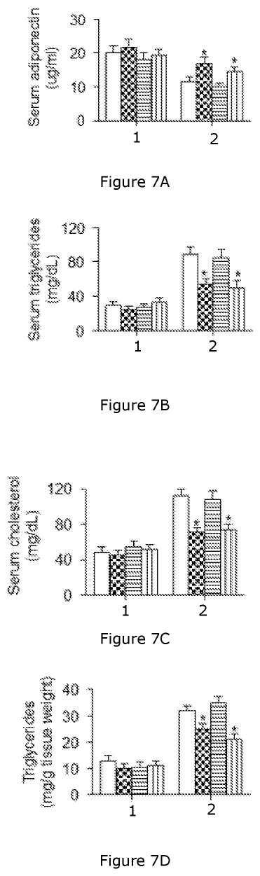

At the end of the four-week treatment, mice were fasted for 16-hours and sacrificed to collect serum and tissue samples for analyses. The circulating concentration of adiponectin was significantly decreased by HFD (by ˜45%) when compared to mice under STC (FIG. 7A). Treatment with either CPP-M1 or CPP-M2 prevented the reduction of adiponectin concentrations in serum (FIG. 7A). When compared to mice under STC, HFD augmented the circulating triglyceride and cholesterol levels, which were significantly reduced by treatment with either CPP-M1 or CPP-M2 (FIG. 7B and 7C). HFD significantly increased the amount of triglyceride accumulated in the liver tissues. Chronic treatment with CPP-M1 or CPP-M2 attenuated the lipid accumulation in livers of mice under HFD (FIG. 7D).

### EXAMPLE 5

This example describes an assay for screening peptides derived from putative

ERp44 binding domains to identify cargo peptides for use in the invention.

### 1. Methods

The peptides listed in Table 3 below, each comprising the TAT CPP and a cargo peptide derived from a putative ERp44 binding domain, will be synthesised according to the methods described above in Example 1.

TABLE 3
Peptides to be screened showing the cargo peptide portion
underlined

| Source protein | Sequence | Seq ID No. |
|---|---|---|
| SERT-C109 | YGRKKRRQQRRRYICYQ | 63 |
| SERT-C200 | YGRKKRRQQRRRTSCKN | 64 |
| SERT-C209 | YGRKKRRQQRRRGNCTN | 65 |
| PRX4-C208 (I) | YGRKKRRQQRRRHGEVCPAGW | 66 |
| PRX4-C208 (II) | YGRKKRRQQRRREVCPA | 67 |
| IL2-C78 | YGRKKRRQQRRRLQCLE | 68 |
| IL2-C125 | YGRKKRRQQRRRFMCEY | 69 |
| IL2-C145 | YGRKKRRQQRRRTFCQS | 70 |
| ERP44-TAIL- | YGRKKRRQQRRRRYCLL | 71 |
| C369 | | |
| ERO1-ALPHA- | YGRKKRRQQRRRSQCGRRDCAVKPCQS | 72 |
| C94-C99-C104 | | |

Murine 3T3L1 adipocytes will be cultured and treated with each peptide as described above in Example 2. The amounts and oligomeric distribution of adiponectin in the conditioned media will be determined using methods described above in Example 2. The interaction of the peptide with ERp44 will be determined by co-immunoprecipitation as described above in Example 2.

The peptides will be administered to mice as described above in Example 3. The effect of the peptides on fasting body weight, body fat mass, serum adiponectin, fasting blood glucose, insulin sensitivity, serum triglycerides, serum cholesterol, and liver triglycerides will be determined as described above in Examples 3 and 4.

### 2. Results

Peptides suitable for use in the invention will
bind ERp44 as determined by co-immunoprecipitation,
induce an increase in secretion of adiponectin and/or a decrease in intracellular adiponectin in adipocytes, and/or
induce a decrease in body fat mass, fasting blood glucose, serum triglycerides, serum cholesterol and/or liver triglycerides and/or an increase in serum adiponectin and/or insulin sensitivity in mice.

### EXAMPLE 6

The purpose of this example is to assess the ERp44 binding profile of peptides of the invention.

### 1. Methods

The binding of the peptides described in Table 2 above to ERp44 at high and low pH will be assessed by in vitro peptide release assay.

Recombinant ERp44 will be produced as described in Example 1.

ERp44 will be incubated for one week at 4° C. with a 10-fold excess of individual peptide in 20mM MES, 150 mM NaCl, pH 6.5 to mimic the low pH in the cis-Golgi where ERp44 binds adiponectin. The pH will be increased to pH 8.0 to mimic the change of pH when the ERp44-adiponectin complex is transported to the endoplasmic reticulum (20 mM Tris-HCl, 150 mM NaCl, pH 8.0) and incubated overnight at 4° C. The samples will be analysed by non-reducing SDS-PAGE and ESI-MS as described in Example 1 to determine the amount of residual ERp44-complex and therefore the degree of peptide release.

### 2. Results

The peptides of the invention will bind ERp44 at low pH to form ERp44-peptide complexes. The degree of complexation is reduced by at least about 20%, 30%, 40%, 50%, 60%, 70% or at least about 80% at high pH.

### EXAMPLE 7

The purpose of this example was to assess the effect of peptides of the invention on adiponectin secretion from adipocytes and adiponectin production in mice fed a high fat diet.

### 1. Methods

The peptides listed in Table 4 below, each comprising the TAT CPP and a cargo peptide derived from a putative ERp44 binding domain, were synthesised according to the methods described above in Example 1.

TABLE 4
Peptides tested showing the cargo peptide portion underlined

| | Source | | | |
|---|---|---|---|---|
| | ERp44 | | | |
| | binding | | Molecular | Yield |
| | domain/ | Sequence/ | Weight | (mg) and |
| Peptide | Mutation | SEQ ID NO | (Da) | purity |
| S-16 | SERT-C200 | YGRKKRRQRRRWTSCKNSW | 2553 | 7.1 mg |
| | | Cargo peptide: SEQ ID NO: 73 | | >95% |
| | | CPP-cargo peptide: SEQ ID NO: 74 | | |
| S-21 | SERT-C209 | YGRKKRRQRRRTGNCTNYF | 2461 | 5.8 mg |
| | | Cargo peptide: SEQ ID NO: 75 | | >95% |
| | | CPP-cargo peptide: SEQ ID NO: 76 | | |
| S-19 | SERT-C200- | YGRKKRRQRRRWTSCKNSWNTGNCTNY | 3421 | 4.2 mg |
| | C209 | Cargo peptide: SEQ ID NO: 77 | | >95% |
| | | CPP-cargo peptide: SEQ ID NO: 78 | | |
| 144-2 | Adiponectin | YGRKKRRQRRRKGTCYGWMA | 2558 | 4.8 mg |
| | (mouse) | Cargo peptide: SEQ ID NO: 79 | | >95% |
| | A→Y | CPP-cargo peptide: SEQ ID NO: 80 | | |
| 148-1 | Adiponectin | YGRKKRRQRRRGTCYGWMA | 2430 | 5.9 mg |
| | (mouse) | Cargo peptide: SEQ ID NO: 81 | | 7.0 mg |
| | A→Y | CPP-cargo peptide: SEQ ID NO: 82 | | >95% |
| | No K | | | |
| 152-1 | IgM | YGRKKRRQRRRGTCA | 1892 | 7.3 mg |
| | Y→A | Cargo peptide: SEQ ID NO: 83 | | >95% |
| | | CPP-cargo peptide: SEQ ID NO: 84 | | |
| 156-1 | IgM | YGRKKRRQRRRKGTCY | 2112 | 6.2 mg |
| | + K | Cargo peptide: SEQ ID NO: 85 | | >95% |
| | | CPP-cargo peptide: SEQ ID NO: 86 | | |
| CPP- | Adiponectin | YGRKKRRQRRRKGTCAGWMA | 2466 | 6.2 mg |
| adiponectin | | Cargo peptide: 59 | | >95% |
| | | CPP-cargo peptide: 60 | | |
| CPP-M1 | IgM | YGRKKRRQRRRGTCY | 1984 | 12.7 mg |
| | | Cargo peptide: 25 | | >95% |
| | | CPP-cargo peptide: 61 | | |

### Cell Culture

3T3-L1 cells (ATCC #CL-173, Manassas, VA, USA) were cultured in Dulbecco's Modified Eagle Medium (DMEM) supplemented with 10% fetal bovine serum (FBS) and 1% penicillin-streptomycin-fungisone (PSF) at 37° C. and under 5% CO2 95% humidified air. For differentiation, the confluent 3T3-L1 cultures were supplemented with 10 pg/ml insulin, 0.25 μM dexamethasone and 0.5 mM isobutylmethylxanthine for two days, followed by incubation with 10 μg/ml insulin in the culture medium for another two days. Subsequently, cells were incubated in DMEM with 10% FBS that was changed every two days. Peptide treatment (200 nM in FBS-free medium) was performed in fully differentiated 3T3-L1 adipocyte cultures. The conditioned medium was collected at 8 and 24 hours after treatment for subsequent analyses of total adiponectin levels using an in house ELISA method as described above in Example 2.

### Animal Studies

Male mice of the C57BL/6J background were housed in a room under controlled temperature (23±1° C.) with 12 hour light-dark cycles and free access to water and food. Mice were fed with a high-fat diet (HFD; 4.73 kcal·g1 containing 45% fat, 20% protein and 35% carbohydrate, D12451, Research diet, N.J., USA), starting from four-weeks old. At the age of eight-weeks, mice were injected with different peptides (10 mg/kg body weight, intraperitoneally). Serum samples were collected from tail vein at 0, 1, 4, 8, 24 and 48 hours after injection.

Total adiponectin levels in the serum samples were determined using methods described above for Example 3.

### 2. Results

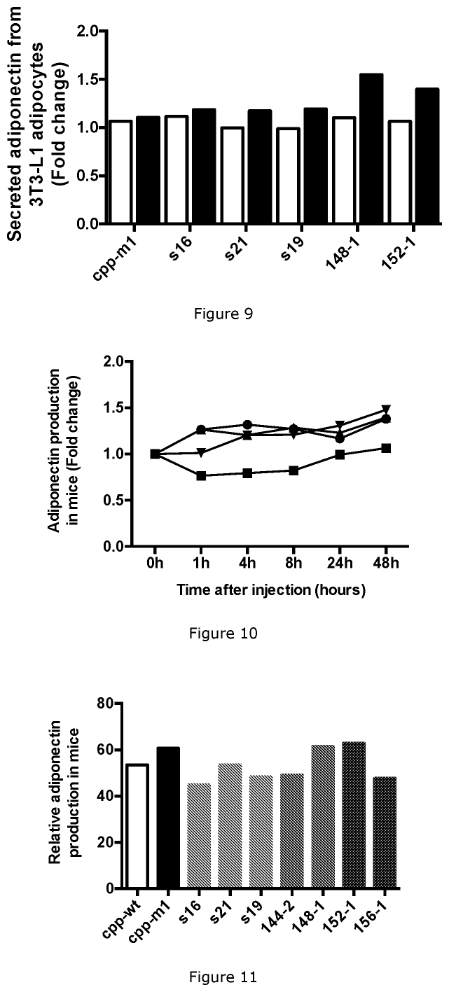

Total adiponectin was measured in the conditioned media of 3T3-L1 adipocytes after treatment with the peptides and fold change relative to untreated controls calculated. Results are shown in FIG. 9.

Total adiponectin was measured over 48 hours in the serum of mice fed a high fat diet following injection of the peptides. Fold change was determined relative to time zero. Curves showing the fold change in serum adiponectin over time for CPP-M1, S-16, 148-1 and 152-1 are shown in FIG. 10. The total area under the time course curve (like those shown in FIG. 10) was calculated for each peptide. Results are shown in FIG. 11.

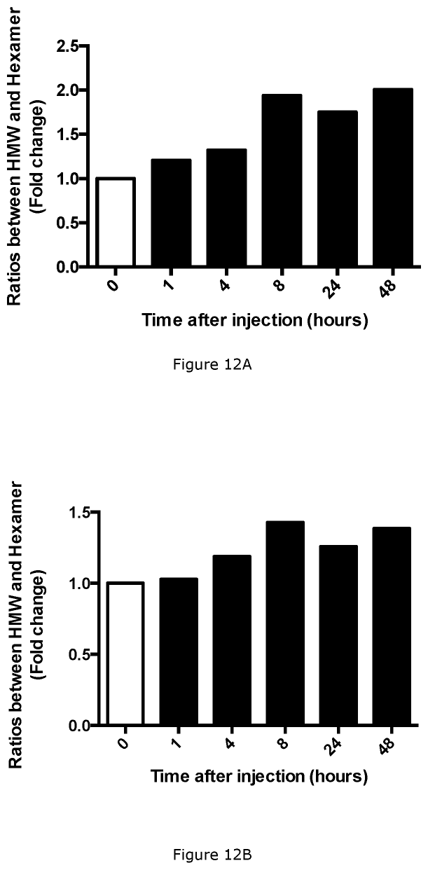

Non-reducing SDS-PAGE was performed to analyze the oligomeric distribution of adiponectin in serum samples from mice treated with peptides 148-1 and 152-1. Results are shown in FIG. 12.

1. A peptide comprising
one or more cargo peptides that bind ERp44, and
one or more cell penetrating agents;
wherein the cargo peptide comprises or consists of the amino acid sequence L¹ -X^{a}-C-X^{b}-L², or a functional variant thereof, wherein
a) X^{a} and X^{b} are each independently 1 to 20 amino acids,
b) L¹ is a linker binding the cell penetrating agent and the cargo peptide, a linker binding the cargo peptide and another cargo peptide or L¹ is absent, and
c) L² is a linker binding the cell penetrating agent and the cargo peptide, a linker binding the cargo peptide and another cargo peptide or L² is absent,
wherein at least one of L¹ and L² is present and is a linker binding the cell penetrating agent and the cargo peptide, and
wherein X^{b} is not AGWMA or X^{b} is not AGWMA for at least one of the cargo peptides if the peptide comprises two or more cargo peptides.

2. A peptide of claim 1 wherein the peptide induces an increase in total secreted adiponectin of at least about 10% as determined by incubating the peptide with 3T3-L1 adipocyte cells for 24 hours at a concentration of 200 nM and measuring total adiponectin in the culture medium of the cells compared to 3T3-L1 adipocyte cells incubated without the peptide under the same conditions.

3. A peptide comprising
one or more cargo peptides, and
one or more cell penetrating agents,
wherein the cargo peptide comprises or consists of the amino acid sequence 12-X^{a}-C-X^{b}-L², or a functional variant thereof, wherein
a) X^{a} and X^{b} are each independently 1 to 20 amino acids,
b) L¹ is a linker binding the cell penetrating agent and the cargo peptide, a linker binding the cargo peptide and another cargo peptide or L¹ is absent, and
c) L² is a linker binding the cell penetrating agent and the cargo peptide, a linker binding the cargo peptide and another cargo peptide or L² is absent,
wherein at least one of L¹ and L² is present and is a linker binding the cell penetrating agent and the cargo peptide, and
wherein the peptide induces an increase in total secreted adiponectin of at least about 10% as determined by incubating the peptide with 3T3-L1 adipocyte cells for 24 hours at a concentration of 200 nM and measuring total adiponectin in the culture medium of the cells compared to 3T3-L1 adipocyte cells incubated without the peptide under the same conditions.

4. A peptide of any one of claims 1 to 3 wherein the cargo peptide comprises or consists of
a) three or more contiguous amino acids from the sequence
[SEQ ID No: 1]
AVDLGNIWRFPYICYQNGGGAF,
b) three or more contiguous amino acids from the sequence
[SEQ ID No: 2]
LISSFTDQLPWTSCKNSWNTGN,
c) three or more contiguous amino acids from the sequence
[SEQ ID No: 3]
PWTSCKNSWNTGNCTNYFAQDN,
d) three or more contiguous amino acids from the sequence
[SEQ ID No: 4]
QAFAQYTDKHGEVCPAGWKPGS,
e) three or more contiguous amino acids from the sequence
[SEQ ID No: 5]
YMPKKATELKHLQCLEEELKP,
f) three or more contiguous amino acids from the sequence
[SEQ ID No: 6]
IVLELKGSETTFMCEYADETAT,
g) three or more contiguous amino acids from the sequence
[SEQ ID No: 7]
ATIVEFLNRWITFCQSIISTLT,
h) three or more contiguous amino acids from the sequence RYCLL [SEQ ID No: 8],
i) three or more contiguous amino acids from the sequence
[SEQ ID No: 9]
DISQCGRRDCAVKPCQSDE,
j) the amino acid sequence YICYQ [SEQ ID No: 10],
k) the amino acid sequence TSCKN [SEQ ID No: 11],
l) the amino acid sequence GNCTN [SEQ ID No: 12],
m) the amino acid sequence HGEVCPAGW [SEQ ID No: 13],
n) the amino acid sequence EVCPA [SEQ ID No: 14],
o) the amino acid sequence LQCLE [SEQ ID No: 15],
p) the amino acid sequence FMCEY [SEQ ID No: 16],
q) the amino acid sequence TFCQS [SEQ ID No: 17],
r) the amino acid sequence SQCGRRDCAVKPCQS [SEQ ID No: 18],
s) three or more contiguous amino acids from the sequence PTLYNVSLVMSDTAGTCY [SEQ ID No: 19],
t) three or more contiguous amino acids from the sequence PTLYNVSLIMSDTGGTCY [SEQ ID No: 20],
u) three or more contiguous amino acids from the sequence
[SEQ ID No: 21]
PTHVNVSVVMAEVDGTCY,
v) three or more contiguous amino acids from the sequence
[SEQ ID No: 22]
PTNVSVVSVIMSEGDGICY,,
w) the amino acid sequence DTAGTCY [SEQ ID No: 23],
x) the amino acid sequence DTGGTCY [SEQ ID No: 24],
y) the amino acid sequence GTCY [SEQ ID No: 25], or
z) a functional variant of any one of a) to y).

5. A peptide of any one of claims 1 to 3 wherein the cargo peptide comprises or consists of
a) the amino acid sequence WTSCKNSW [SEQ ID No:73],
b) the amino acid sequence TGNCTNYF [SEQ ID No:75],
c) the amino acid sequence WTSCKNSWNTGNCTNY [SEQ ID No: 77],
d) the amino acid sequence GTCA [SEQ ID No: 83],
e) the amino acid sequence GTCYGWMA [SEQ ID No: 81], or
f) the amino acid sequence GTCAGWMA [SEQ ID No: 87] or
g) a functional variant of any one of a) to f).

6. A peptide of any one of claims 1 to 5 comprising
one or more cargo peptides, and
one or more cell penetrating agents;
wherein the cargo peptide comprises or consists of the amino acid sequence L¹-X¹-X²-X³-C-X⁴-X⁵-L² or a functional variant thereof, wherein
a) X¹ is absent or is 1 to 20 amino acids,
b) X² is selected from the group comprising glycine, alanine, valine, leucine, isoleucine, methionine, proline, phenylalanine and tryptophan,
c) X³ is selected from the group comprising glycine, alanine, valine, leucine, isoleucine, methionine, proline, phenylalanine and tryptophan, serine, threonine, asparagine, glutamine, tyrosine, cysteine, lysine, arginine, histidine, aspartic acid and glutamic acid,
d) X⁴ is selected from the group comprising serine, threonine, asparagine, glutamine, tyrosine, cysteine, lysine, arginine, histidine, aspartic acid, glutamic acid, phenylalanine and tryptophan,
e) X⁵ is absent or is 1-20 amino acids,
f) L¹ comprises a linker binding the cell penetrating agent and the cargo peptide, a linker binding the cargo peptide and another cargo peptide or L¹ is absent, and
g) L² comprises a linker binding the cell penetrating agent and the cargo peptide, a linker binding the cargo peptide and another cargo peptide or L² is absent,
wherein at least one of L¹ and L² is present and is a linker binding the cell penetrating agent and the cargo peptide.

7. A peptide of any one of claims 1 to 5 comprising
one or more cargo peptides, and
one or more cell penetrating agents;
wherein the cargo peptide comprises or consists of the amino acid sequence L¹-X¹-X²-X³-C-X⁴-X⁵-L² or a functional variant thereof, wherein
a) X¹ is absent or is 1 to 20 amino acids,
b) X² is selected from the group comprising glycine, alanine, valine, leucine, isoleucine, methionine, proline, phenylalanine, threonine, serine and tryptophan,
c) X³ is selected from the group comprising glycine, alanine, valine, leucine, isoleucine, methionine, proline, phenylalanine and tryptophan, serine, threonine, asparagine, glutamine, tyrosine, cysteine, lysine, arginine, histidine, aspartic acid and glutamic acid,
d) X⁴ is selected from the group comprising serine, threonine, asparagine, glutamine, tyrosine, cysteine, lysine, arginine, histidine, aspartic acid, glutamic acid, phenylalanine, alanine, glycine, valine and tryptophan,
e) X⁵ is absent or is 1-20 amino acids,
f) L¹ comprises a linker binding the cell penetrating agent and the cargo peptide, a linker binding the cargo peptide and another cargo peptide or L¹ is absent, and
g) L² comprises a linker binding the cell penetrating agent and the cargo peptide, a linker binding the cargo peptide and another cargo peptide or L² is absent,
wherein at least one of L¹ and L² is present and is a linker binding the cell penetrating agent and the cargo peptide.

8. A peptide of claim 6 or 7 wherein X² is selected from the group comprising
a) glycine, alanine, valine and leucine, or
b) glycine and alanine.

9. A peptide of claim 6 or 7 wherein X² is selected from the group comprising
a) glycine, alanine, valine, threonine, serine and leucine,
b) glycine, threonine, serine and alanine, or
c) glycine or threonine.

10. A peptide of any one of claims 6 to 9 wherein X³ is selected from the group comprising
a) serine, threonine, asparagine, glutamine, tyrosine, cysteine, lysine, arginine, histidine, aspartic acid and glutamic acid,
b) serine, threonine, asparagine, glutamine, cysteine and tyrosine,
c) serine, threonine, asparagine, and glutamine,
d) threonine, serine and tyrosine, or
e) threonine and serine.

11. A peptide of any one of claims 6 to 10 wherein X⁴ is selected from the group comprising
a) serine, threonine, asparagine, glutamine, tyrosine, cysteine, lysine, arginine, histidine, aspartic acid and glutamic acid,
b) serine, threonine, asparagine, glutamine, tyrosine and cysteine, or
c) tyrosine, phenylalanine and tryptophan.

12. A peptide of any one of claims 7 to 11 wherein X⁴ is selected from the group comprising
a) serine, threonine, asparagine, glutamine, tyrosine, cysteine, lysine, arginine, histidine, aspartic acid, alanine, glycine, valine and glutamic acid,
b) serine, threonine, asparagine, glutamine, tyrosine, alanine, valine, glycine and cysteine,
c) tyrosine, phenylalanine, alanine, glycine, valine and tryptophan, and
d) tyrosine and alanine.

13. A peptide of any one of claims 1 to 12 wherein the linker comprises a peptide bond, one or more amino acids, a covalent bond, or a chemical spacer.

14. A peptide of any one of claims 1 to 13 wherein the peptide binds ERp44 at a pH of about 6.5 as determined by incubating ERp44 with a 10 fold excess of the peptide for one week at pH 6.5 and detecting ERp44-peptide interaction by electrospray ionization mass spectrometry.

15. A peptide of any one of claims 1 to 14 wherein the cell penetrating agent comprises a cell penetrating peptide.

16. A peptide of any one of claims 1 to 15 wherein the cell penetrating peptide is selected from the group comprising a cationic cell penetrating peptide, an amphipathic cell penetrating peptide and a hydrophobic cell penetrating peptide.

17. A peptide of any one of claims 1 to 16 wherein the cell penetrating peptide is a cyclic peptide.

18. A peptide of any one of claims 1 to 17 wherein the cell penetrating peptide is selected from the group consisting of TAT, Penetratin (Antp), DPV1047, Bac, SynB1, SynB1-NLS, Poly-arginine, VP22, Transportan, MAP, pVEC, MTS, hCT derived, MPG, Buforin 2, PEP-1, Magainin 2, M918 C105Y, PFVYLI, BPrPR(1-28), ARF(1-22), p28, VT5, YTA2, YTA4, CADY and PEP-7.

19. A peptide of any one of claims 1 to 14 wherein the cell penetrating agent is a cell penetrating peptoid.

20. A peptide of claim 19 wherein the cell penetrating peptoid is an oligomer of N-substituted glycine units.

21. A pharmaceutical composition comprising a peptide of any one of claims 1 to 20 and a pharmaceutically acceptable carrier.

22. A method of treating or preventing a condition associated with adiponectin dysfunction the method comprising administering to the subject a therapeutically effective amount of a peptide or pharmaceutical composition of any one of claims 1 to 21.

23. A method of claim 22 wherein the condition associated with adiponectin dysfunction is a condition associated with reduced serum adiponectin, reduced insulin sensitivity, increased serum cholesterol, increased serum triglycerides, or increased blood glucose.

24. A method of claim 22 or 23 wherein the condition associated with adiponectin dysfunction is metabolic syndrome, insulin resistance, diabetes, musculoskeletal disease, musculoskeletal disease, cardiovascular disease, respiratory disease, gallbladder disease, liver disease, gynaecological disease, sexual dysfunction, neurodegenerative disease or cancer.

25. A method of any one of claims 22 to 24 wherein the condition associated with adiponectin dysfunction is type 2 diabetes, hypertension, hypercholesterolemia, hypertriglyceridemia, osteoarthritis, atherosclerosis, heart attack, stroke, sleep apnoea, nonalcoholic fatty liver disease, infertility, irregular menstruation, erectile dysfunction, Alzheimer's disease, Parkinson's disease, endometrial cancer, uterine cancer, breast cancer, ovarian cancer, prostate cancer, liver cancer, gallbladder cancer, kidney cancer, rectal cancer, oesophageal cancer, gallbladder cancer or colon cancer.
